(19) United States
(12) Patent Application Publication
(10) Pub. No.: US 2021/0024944 A1
(43) Pub. Date: Jan. 28, 2021

(54) METHOD OF PRODUCING ALKALOID

(71) Applicant: AJINOMOTO CO., INC., Tokyo (JP)

(72) Inventors: Hiroaki Kisaka, Kawasaki-shi (JP); Tetsuya Miwa, Kawasaki-shi (JP); Hiroto Hirano, Kawasaki-shi (JP)

(73) Assignee: AJINOMOTO CO., INC., Tokyo (JP)

(21) Appl. No.: 17/062,155

(22) Filed: Oct. 2, 2020

Related U.S. Application Data
(63) Continuation of application No. PCT/JP2019/015184, filed on Apr. 5, 2019.

(30) Foreign Application Priority Data
Apr. 6, 2018 (JP) 2018-073800

(51) Int. Cl. C12N 15/82

(52) U.S. Cl. CPC C12N 15/8243

(57) ABSTRACT

A production method can produce alkaloids at higher efficiencies than conventional methods includes expressing, in a Catharanthus plant of the family Apocynaceae and using gene manipulation, a protein that particulates in tryptophan biosynthesis and a protein that participates in secologanin biosynthesis.

This application is a Continuation of, and claims priority under 35 U.S.C. § 120 to, International Application No. PCT/JP2019/015184, filed Apr. 5, 2019, and claims priority therethrough under 35 U.S.C. §§ 119, 365 to Japanese Patent Application No. 2018-073800, filed Apr. 6, 2018, the entireties of which are incorporated by reference herein. Also, the Sequence Listing filed electronically herewith is hereby incorporated by reference (File name: 2020-10-02T_US-622_Seq_List; File size: 18 KB; Date recorded: Oct. 2, 2020).

### TECHNICAL FIELD

The present invention relates to a method of producing alkaloids.

### BACKGROUND

Vinca alkaloids such as vinblastine and vincristine are monoterpene indole alkaloids (MIA) extracted from plants of the genus Catharanthus in the family Apocynaceae, and are known to exhibit potent microtubule polymerization inhibitory activity. They are also marketed as an anticancer agent for Hodgkin's disease, malignant lymphoma, and the like.

It can be said that plants of the genus Catharanthus in the family Apocynaceae grow faster than trees and have less environmental problems, and are therefore excellent as raw materials for the production of MIA. However, since the amount of alkaloids obtained by the extraction of the plants of the genus Catharanthus in the family Apocynaceae is very small (about 100 μg/gFW in the case of vinca alkaloids), it is impossible to meet the expected increase in demand in the future unless the plant is cultivated in large quantities.

As a method of increasing the production amount of alkaloids in plants of the genus Catharanthus in the family Apocynaceae, Non Patent Literature 1 states that the use of a glucocorticoid-inducible promoter to express, in hairy roots of Catharanthus roseus, the anthranilate synthase a subunit having resistance to feedback inhibition by tryptophan derived from Arabidopsis increases the yield of tryptophan and tryptamine, which thereby increases the amount of lochnericine, a type of monoterpene indole alkaloid (MIA).

In addition, Non Patent Literature 2 states that overexpression of ORCA4 increases the amount of accumulated vinca alkaloid biosynthesis intermediates of the hairy roots of Catharanthus roseus (page 1108, right column, lines 6 to 7).

### CITATION LIST

### Non Patent Literature

Non Patent Literature 1: Hughes et al., Expression of a Feedback-Resistant Anthranilate Synthase in Catharanthus roseus Hairy Roots Provides Evidence for Tight Regulation of Terpenoid Indole Alkaloid Levels, Biotechnology and Bioengineering, Vol. 86, No. 6, Jun. 20, 2004, pp. 718-727

Non Patent Literature 2: Paul et al., A differentially regulated AP2/ERF transcription factor gene cluster acts downstream of a MAP kinase cascade to modulate terpenoid indole alkanoid biosynthesis in Catharanthus roseus, New Phytologist (2017) 213: pp. 1107-1123

### SUMMARY

The method of Non Patent Literature 1 has a problem that the expression is limited to hairy roots, and the amount of terpenoids other than lochnericine does not significantly increase (Abstract of Non Patent Literature 1). The method of Non Patent Literature 2 is similarly a finding limited to hairy roots. Although the production efficiency of alkaloids is improved to some extent by overexpression of ORCA4, it cannot be said yet that the production efficiency is sufficient, considering the expected increase in demand in the future. Therefore, there is a great demand for a production method having higher production efficiency of alkaloids than ever before.

The subject matter of the present disclosure has been made in view of the above problems, and makes it possible to produce alkaloids with higher efficiency than ever before by introducing a gene encoding a protein involved in tryptophan biosynthesis and a gene encoding a protein involved in secologanin biosynthesis into plants of the genus Catharanthus in the family Apocynaceae. This disclosure includes the following configurations.

A method of producing an alkaloid, comprising: expressing, using genetic engineering, a protein involved in tryptophan biosynthesis and a protein involved in secologanin biosynthesis in a plant of a genus Catharanthus in a family Apocynaceae.

A method of producing an alkaloid as above, wherein the proteins are expressed in the plant of the genus Catharanthus in the family Apocynaceae by introducing a gene encoding the protein involved in tryptophan biosynthesis and a gene encoding the protein involved in secologanin biosynthesis into the plant of the genus Catharanthus in the family Apocynaceae.

A method as above, wherein the proteins are expressed in the plant of the genus Catharanthus in the family Apocynaceae by introducing the gene encoding the protein involved in tryptophan biosynthesis and the gene encoding the protein involved in secologanin biosynthesis into the plant of the genus Catharanthus in the family Apocynaceae using a vector containing the genes.

A method as above, wherein the proteins are expressed in the plant of the genus Catharanthus in the family Apocynaceae by introducing the gene encoding the protein involved in tryptophan biosynthesis and the gene encoding the protein involved in secologanin biosynthesis into a leaf and/or stem of Catharanthus roseus.

A method as above, wherein the protein involved in secologanin biosynthesis is ORCA4.

A method as above, wherein the protein involved in tryptophan biosynthesis contains at least one protein selected from the group consisting of AroG, TrpD, and TrpE.

A method as above, wherein the protein involved in tryptophan biosynthesis contains AroG, TrpD, and TrpE.

A method as above, wherein
the AroG is an AroG protein modified to suppress feedback control of 3-deoxy-D-arabino-heptulosonate-7-phosphate synthase by phenylalanine, and/or
the TrpE is a TrpE protein modified to suppress feedback control of anthranilate synthase by tryptophan.

A method as above, wherein
the AroG is an AroG protein obtained by expressing a gene having a DNA sequence of SEQ ID NO: 27, and/or
the TrpD is a TrpD protein obtained by expressing a gene having a DNA sequence of SEQ ID NO: 28, and/or
the TrpE is a TrpE protein obtained by expressing a gene having a DNA sequence of SEQ ID NO: 30.

A method as above, wherein the alkaloid is a vinca alkaloid.

A method as above, wherein the alkaloid is at least one alkaloid selected from the group consisting of tabersonine, vindoline, catharanthine, vinblastine, vincristine, ajmalicine, serpentine, stemmadenine, and strictosidine.

A method as above, wherein the alkaloid is tabersonine.

A method as above, wherein the alkaloid is stemmadenine.

A method as above, wherein the plant is Catharanthus roseus.

A vector comprising: a gene encoding a protein involved in tryptophan biosynthesis and a gene encoding a protein involved in secologanin biosynthesis.

An Agrobacterium comprising:
a vector containing a gene encoding a protein involved in tryptophan biosynthesis and a gene encoding a protein involved in secologanin biosynthesis, or
a mixture of vectors containing at least one gene selected from the gene encoding the protein involved in tryptophan biosynthesis and the gene encoding the protein involved in secologanin biosynthesis, the vector mixture containing the gene encoding the protein involved in tryptophan biosynthesis and the gene encoding the protein involved in secologanin biosynthesis in the entire mixture.

A plant of a genus Catharanthus in a family Apocynaceae, the plant comprising:
a vector containing a gene encoding a protein involved in tryptophan biosynthesis and a gene encoding a protein involved in secologanin biosynthesis, or
a mixture of vectors containing at least one gene selected from the gene encoding the protein involved in tryptophan biosynthesis and the gene encoding the protein involved in secologanin biosynthesis, the vector mixture containing the gene encoding the protein involved in tryptophan biosynthesis and the gene encoding the protein involved in secologanin biosynthesis in the entire mixture.

A plant of a genus Catharanthus in a family Apocynaceae, which has been transformed so as to overproduce alkaloids by introducing
a vector containing a gene encoding a protein involved in tryptophan biosynthesis and a gene encoding a protein involved in secologanin biosynthesis, or
a mixture of vectors containing at least one gene selected from the gene encoding the protein involved in tryptophan biosynthesis and the gene encoding the protein involved in secologanin biosynthesis, the vector mixture containing the gene encoding the protein involved in tryptophan biosynthesis and the gene encoding the protein involved in secologanin biosynthesis in the entire mixture.

The subject matter of the present disclosure may make it possible to produce alkaloids with high efficiency.

### BRIEF DESCRIPTION OF DRAWINGS

FIG. 1 is a diagram explaining an alkaloid synthesis pathway in Catharanthus roseus.

FIG. 2 is a design diagram of each of the vectors used in Examples of the present disclosure.

FIG. 3 is a diagram illustrating the transcriptional expression levels of (A) aroG4 gene, (B) trpE8 gene, and (C) trpD gene by Real-Time PCR analysis in the case of introducing pRI201-AN, AED, ORCA4, or AED-ORCA4. In the figure, pRI201-AN represents a vector control, AED represents an aroG4-trpE8-trpD gene linked vector, ORCA4 represents a vector containing the ORCA4 gene, and AED-ORCA4 represents an aroG4-trpE8-trpD-ORCA4 gene linked vector. Note that the number of samples is 3 (n=3).

FIG. 4 is a diagram illustrating the transcriptional expression level of the ORCA4 gene by Real-Time PCR analysis in the case of introducing pRI201-AN, AED, ORCA4, or AED-ORCA4. FIG. 4A illustrates the transcriptional expression level of the introduced ORCA4 gene, and FIG. 4B illustrates the transcriptional expression level of the endogenous ORCA4 gene. Note that the number the number of samples is 3 (n=3).

FIG. 5 is a diagram illustrating the tryptophan content in Catharanthus roseus in the case of introducing pRI201-AN, AED, ORCA4, or AED-ORCA4. Note that the number of samples is 3 (n=3).

FIG. 6 is a diagram illustrating the tabersonine content in Catharanthus roseus in the case of introducing pRI201-AN, AED, ORCA4, or AED-ORCA4. Note that the number of samples is 3 (n=3).

FIG. 7 is a diagram illustrating the stemmadenine content in Catharanthus roseus in the case of introducing pRI201-AN, AED, ORCA4, or AED-ORCA4. Note that the number of samples is 3 (n=3).

FIG. 8 is the DNA sequence of the aroG gene derived from Escherichia coli.

FIG. 9 is the DNA sequence of the aroG4 gene obtained by modifying the aroG gene derived from Escherichia coli.

FIG. 10 is the DNA sequence of the trpD gene derived from Escherichia coli.

FIG. 11 is the DNA sequence of the trpE gene derived from Escherichia coli.

FIG. 12 is the DNA sequence of the trpE8 gene obtained by modifying the trpE gene derived from Escherichia coli.

FIG. 13 is the DNA sequence of the chloroplast transfer signal of aldolase derived from Catharanthus roseus.

FIG. 14 is the DNA sequence of the ORCA4 gene derived from Catharanthus roseus.

FIG. 15 is an RNAi vector constructed to suppress the expression of mRNA of the PAS gene, the CS gene, and the TS gene.

FIG. 16 is a base sequence (293 bp) of the PAS gene used for preparing the PAS-RNAi vector.

FIG. 17 is a base sequence (254 bp) of the CS gene used for preparing the CS-RNAi vector.

FIG. 18 is a base sequence (257 bp) of the TS gene used for preparing the TS-RNAi vector.

### DETAILED DESCRIPTION

A first aspect of the present disclosure includes a method of producing an alkaloid, including: expressing, using genetic engineering, a protein (or a polypeptide) involved in tryptophan biosynthesis and a protein (or a polypeptide) involved in secologanin biosynthesis in a plant of a genus Catharanthus in a family Apocynaceae.

The biosynthetic pathway of tryptophan (L-tryptophan) in the plant is described. L-tryptophan is biosynthesized from chorismic acid, a common intermediate for aromatic amino acids. L-tryptophan is biosynthesized by the action of five kinds of enzymes synthesized by the tryptophan operon trpEDCBA on chorismic acid produced through a shared shikimic acid pathway starting from phosphoenolpyruvic acid and D-erythrose-4-phosphate. First, anthranilate synthase (TrpE-TrpD complex) acts on chorismic acid to synthesize anthranilic acid, and then anthranilate phosphoribosyl transferase (TrpD) acts to synthesize phosphoribosyl anthranilate. Then, phosphoribosyl anthranilate isomerase (TrpC) and indole-3-glycerol phosphate synthase (TrpC) act to synthesize indole-3-glycerol phosphate, where tryptophan synthase (TrpB-TrpA complex) acts to complete the synthesis of L-tryptophan (Bonggaerts et al., MetabEng, 3, 289-300, 2001).

As described above, various proteins (enzymes) are involved in tryptophan biosynthesis, and the amount of tryptophan synthesized can be increased by increasing the amounts of these enzymes in vivo.

In addition, tryptophan is, when its synthesis amount increases, known to perform negative feedback control by binding tryptophan to the a subunit (TrpE) of anthranilate synthase. Therefore, the amount of tryptophan synthesized can be further increased by modifying the a subunit (trpE) of anthranilate synthase so that it cannot bind to tryptophan.

1. Protein Involved in Tryptophan Biosynthesis

In methods of the present disclosure, a protein involved in tryptophan biosynthesis is expressed, using genetic engineering, in a plant of the genus Catharanthus in the family Apocynaceae.

The protein involved in tryptophan biosynthesis includes anthranilate synthase (trpE), phosphoglycerate dehydrogenase (SerA), 3-deoxy-D-arabino heptulosonate-7-phosphate synthase (AroG), 3-dehydroquinate synthase (AroB), shikimate dehydrogenase (AroE), shikimate kinase (AroL), 5-enolpyruvylshikimate-3-phosphate synthase (AroA), chorismate synthase (AroC), prephenate dehydratase, chorismate mutase (PheA), tryptophan synthase (TrpA, TrpB), phosphoribosyl anthranilate isomerase (TrpC), indole-3-glycerol phosphate synthase (TrpC), and anthranilate phosphoribosyl transferase (TrpD). Note that the protein may be modified so that its enzymatic activity is increased. In addition, the a subunit (TrpE) of anthranilate synthase known to undergo negative feedback control by binding to tryptophan, and 3-deoxy-D-arabino heptulosonate-7-phosphate synthase (AroG) known to undergo negative feedback control by binding to phenylalanine may be modified so that the negative feedback control is reduced or modified so that the control is released.

Among these, the protein involved in tryptophan biosynthesis preferably contains at least one protein selected from AroG, TrpD, and TrpE, contains a combination of TrpD and TrpE and/or AroG, or contains AroG, TrpD, and TrpE. In addition, TrpE may be a TrpE protein modified to suppress feedback control of anthranilate synthase by tryptophan. AroG may be an AroG protein modified to suppress feedback control of 3-deoxy-D-arabino-heptulosonate-7-phosphate synthase by phenylalanine.

Description of AroG, TrpD, and TrpE

AroG Protein

AroG is an enzyme involved in the synthesis of the first precursor, 3-deoxy-D-arabino heptulosonate 7 phosphate synthase (NAHP), of aromatic amino acids during tryptophan biosynthesis, namely 3-deoxy-7-phosphoheptulonate synthase (AroG). The action of the enzyme synthesizes NAHP from phosphoenolpyruvic acid and D-erythrose 4-phosphate, and furthermore, NAHP is metabolized into chorismic acid through several metabolic processes, which ultimately increases the amount of tryptophan biosynthesis. The majority of plants and microorganisms are applicable as organisms having a gene (aroG) encoding the enzyme, and examples thereof include E. coli, Corynebacterium glutamicum, Bacillus subtilis, Arabidopsis thaliana, Oryza sativa, and the like. In the present disclosure, AroG may be a plant-derived and/or microorganism-derived AroG protein. Preferably, AroG is derived from Escherichia coli (E. coli).

In the present disclosure, AroG may have or need not have a modification in the amino acid sequence as long as it functions as a 3-deoxy-7-phosphoheptulonate synthase. AroG may have 90% or more homology with the amino acid sequence of AroG possessed by any of the above plants or microorganisms (preferably E. coli), 95% or more homology with the sequence, 98% or more homology with the sequence, 99% or more homology with the sequence, or 100% homology with the sequence.

The above variant of AroG may be modified so that the feedback control by phenylalanine is released. By expressing the variant in a plant by genetic engineering, negative feedback control does not occur even when the amount of tryptophan biosynthesis increases, so that the amount of tryptophan biosynthesis can be further increased. As such AroG protein, AroG4, AroG15, and the like are known. For AroG4, it is possible to refer to APPLIED AND ENVIRONMENTAL MICROBIOLOGY February 1997, p. 761-762. The same reference can be made also for AroG15.

The AroG protein expressed in a plant of the present disclosure may be an AroG protein obtained by expressing a gene having a DNA sequence having 90% or more homology with the DNA sequence set forth in SEQ ID NO: 26 or 27 derived from Escherichia coli, an AroG protein obtained by expressing a gene having a DNA sequence having 95% or more homology with the sequence, an AroG protein obtained by expressing a gene having a DNA sequence having 98% or more homology with the sequence, an AroG protein obtained by expressing a gene having a DNA sequence having 99% or more homology with the sequence, an AroG protein obtained by expressing a gene having a DNA sequence having 99.5% or more homology with the sequence, an AroG protein obtained by expressing a gene having a DNA sequence having 99.9% or more homology with the sequence, an AroG protein obtained by expressing a gene having the DNA sequence set forth in SEQ ID NO: 26 or 27, or an AroG protein obtained by expressing a gene having the DNA sequence set forth in SEQ ID NO: 27 (aroG4).

TrpD Protein

TrpD is anthranilate phosphoribosyl transferase (TrpD). The action of the enzyme synthesizes pyrophosphoric acid and anthranilic acid from anthranilic acid and 5-phosphoribosyl diphosphate, and the synthesized anthranilic acid is metabolized through several metabolic processes, which ultimately increases the amount of tryptophan biosynthesis. The majority of plants and microorganisms are applicable as organisms having a gene encoding the protein, and examples thereof include E. coli, Corynebacterium glutamicum, Bacillus subtilis, Arabidopsis thaliana, Oryza sativa, and the like. In the present disclosure, TrpD may be a plant- and/or microorganism-derived TrpD protein. Preferably, TrpD is a TrpD protein derived from Escherichia coli (E. coli).

In the present disclosure, TrpD may have or need not have a modification in the amino acid sequence as long as it functions as an anthranilate phosphoribosyl transferase. TrpD may have 90% or more homology with the amino acid sequence of TrpD protein possessed by any of the above plants or microorganisms (preferably E. coli), 95% or more homology with the sequence, 98% or more homology with the sequence, 99% or more homology with the sequence, or 100% homology with the sequence.

The TrpD expressed in a plant in the present invention may be a TrpD protein obtained by expressing a gene having a DNA sequence having 90% or more homology with the DNA sequence set forth in SEQ ID NO: 28 derived from Escherichia coli, a TrpD protein obtained by expressing a gene having a DNA sequence having 95% or more homology with the sequence, a TrpD protein obtained by expressing a gene having a DNA sequence having 98% or more homology with the sequence, a TrpD protein obtained by expressing a gene having a DNA sequence having 99% or more homology with the sequence, a TrpD protein obtained by expressing a gene having a DNA sequence having 99.5% or more homology with the sequence, a TrpD protein obtained by expressing a gene having a DNA sequence having 99.9% or more homology with the sequence, or a TrpD protein obtained by expressing a gene having the DNA sequence set forth in SEQ ID NO: 28.

TrpE Protein

TrpE is a protein constituting the a subunit (TrpE) of anthranilate synthase. The protein synthesizes anthranilic acid, glutamic acid, and pyruvic acid from chorismic acid and glutamine, and the synthesized anthranilic acid is metabolized through several metabolic processes, which ultimately increases the amount of tryptophan biosynthesis. The majority of plants and microorganisms are applicable as organisms having a gene encoding the protein, and examples thereof include E. coli, Corynebacterium glutamicum, Bacillus subtilis, Arabidopsis thaliana, Oryza sativa, and the like. In the present disclosure, TrpE may be a plant- and/or microorganism-derived TrpE protein. Preferably, TrpE is a TrpE protein derived from Escherichia coli (E. coli).

In the present disclosure, TrpE may have a modification in the amino acid sequence as long as it functions as the a subunit of anthranilate synthase and acts as anthranilate synthase together with the β subunit. TrpE may have 90% or more homology with the amino acid sequence of TrpE possessed by any of the above plants or microorganisms (preferably E. coli), 95% or more homology with the sequence, 98% or more homology with the sequence, 99% or more homology with the sequence, or 100% homology with the sequence.

The above variant of TrpE is preferably modified to suppress the feedback control of anthranilate synthase by tryptophan. As such TrpE, TrpE8, TRP4, and the like are known. For TrpE8, it is possible to refer to WO 94/08031 and Published Japanese Translation of PCT International Application No. Hei 7-507693. For TRP4, it is possible to refer to THE PLANT CELL 1993 VOL 5, p 1011-1027.

The TrpE expressed in a plant in the present disclosure may be a TrpE protein obtained by expressing a gene having a DNA sequence having 90% or more homology with the DNA sequence set forth in SEQ ID NO: 29 or 30 derived from Escherichia coli, a TrpE protein obtained by expressing a gene having a DNA sequence having 95% or more homology with the sequence, a TrpE protein obtained by expressing a gene having a DNA sequence having 98% or more homology with the sequence, a TrpE protein obtained by expressing a gene having a DNA sequence having 99% or more homology with the sequence, a TrpE protein obtained by expressing a gene having a DNA sequence having 99.5% or more homology with the sequence, a TrpE protein obtained by expressing a gene having a DNA sequence having 99.9% or more homology with the sequence, a TrpE protein obtained by expressing a gene having the DNA sequence set forth in SEQ ID NO: 29 or 30, or a TrpE protein obtained by expressing a gene having the DNA sequence set forth in SEQ ID NO: 30 (trpE8).

2. Protein Involved in Secologanin Biosynthesis

In the method of the present disclosure, a protein involved in secologanin biosynthesis is expressed, using genetic engineering, in a plant of the genus Catharanthus in the family Apocynaceae.

Examples of the protein involved in secologanin biosynthesis include ORCA4, secologanin synthase, loganate methyltransferase, iridoid synthase, and geraniol-10-dehydrogenase. Among these, ORCA4 is preferable.

ORCA4 Protein

ORCA (octadecanoid derivative-Responsive Catharanthus AP2-domain) 4 is a protein presumed to have a master regulator function of an enzyme gene involved in the synthesis of Catharanthus roseus isoprenoid, and is known to be involved in secologanin biosynthesis. When the ORCA4 protein is expressed, the synthesis amount of secologanin synthase (SLS) and loganate methyltransferase (LAMT) increases (Non Patent Literature 2).

The ORCA4 is preferably an ORCA4 protein derived from Catharanthus roseus. The ORCA4 protein derived from Catharanthus roseus may be an ORCA4 protein obtained by expressing a gene having a DNA sequence having 90% or more homology with the DNA sequence set forth in SEQ ID NO: 32, an ORCA4 protein obtained by expressing a gene having a DNA sequence having 95% or more homology with the sequence, an ORCA4 protein obtained by expressing a gene having a DNA sequence having 98% or more homology with the sequence, an ORCA4 protein obtained by expressing a gene having a DNA sequence having 99% or more homology with the sequence, an ORCA4 protein obtained by expressing a gene having a DNA sequence having 99.5% or more homology with the sequence, an ORCA4 protein obtained by expressing a gene having a DNA sequence having 99.9% or more homology with the sequence, or an ORCA4 protein obtained by expressing a gene having the DNA sequence set forth in SEQ ID NO: 32.

3. Protein Expression Method

There is no particular limitation on the method of expressing, using genetic engineering, a protein involved in tryptophan biosynthesis and a protein involved in secologanin biosynthesis in a plant of the genus Catharanthus in the family Apocynaceae. Examples include a method in which the proteins are expressed in the plant of the genus Catharanthus in the family Apocynaceae by introducing a gene encoding the protein involved in tryptophan biosynthesis and a gene encoding the protein involved in secologanin biosynthesis into the plant. Introduction methods include a method of introducing the above genes into the plant using the Agrobacterium method or the particle gun method to induce transformation, and thereby causing the above proteins to be constantly expressed in the plant, a method of introducing the above genes into the plant by the infiltration method or the agroinfection method using a plant virus vector or Agrobacterium, and thereby transiently expressing the above genes in the plant, or the like.

Preferable among these is a method in which the proteins are expressed in the plant of the genus Catharanthus in the family Apocynaceae by introducing a gene encoding the protein involved in tryptophan biosynthesis and a gene encoding the protein involved in secologanin biosynthesis into the plant. In addition, the gene encoding a protein involved in tryptophan biosynthesis may be a gene that increases the amount of tryptophan synthesis when introduced into the plant of the genus Catharanthus in the family Apocynaceae. The gene encoding a protein involved in secologanin biosynthesis may be a gene that increases the amount of secologanin synthesis when introduced into the plant of the genus Catharanthus in the family Apocynaceae.

In the present disclosure, the gene that increases the amount of tryptophan synthesis when introduced into the plant of the genus Catharanthus in the family Apocynaceae means a gene that encodes proteins such as enzymes and transcription factors involved in tryptophan biosynthesis, and can increase the amount of tryptophan synthesis in the plant of the genus Catharanthus in the family Apocynaceae by introducing the gene into the plant and expressing it.

In the present disclosure, the gene that increases the amount of secologanin synthesis when introduced into the plant of the genus Catharanthus in the family Apocynaceae means a gene that encodes proteins such as transcription factors, enzymes, and transporters involved in secologanin biosynthesis, and can increase the amount of secologanin synthesis in the plant of the genus Catharanthus in the family Apocynaceae by introducing the gene into the plant and expressing it.

The extent of protein expression can be evaluated directly by confirming the amount of protein produced. Alternatively, it can be indirectly evaluated by confirming the transcription amount (mRNA amount) of the gene encoding the protein.

4. Gene Encoding Protein Involved in Tryptophan Biosynthesis

Examples of the gene encoding the protein involved in tryptophan biosynthesis include genes encoding anthranilate synthase (trpE), phosphoglycerate dehydrogenase (serA), 3-deoxy-D-arabino heptulosonate-7-phosphate synthase (aroG), 3-dehydroquinate synthase (aroB), shikimate dehydrogenase (aroE), shikimate kinase (aroL), 5-enolpyruvylshikimate-3-phosphate synthase (aroA), chorismate synthase (aroC), prephenate dehydratase, chorismate mutase (pheA), tryptophan synthase (trpA, trpB), phosphoribosyl anthranilate isomerase (TrpC), indole-3-glycerol phosphate synthase (TrpC), and anthranilate phosphoribosyl transferase (TrpD). Note that these genes may be modified so that the enzyme activity of the protein (enzyme) encoded by the gene is increased. In addition, the genes encoding the a subunit (trpE) of anthranilate synthase known to undergo negative feedback control by binding to tryptophan, and 3-deoxy-D-arabino heptulosonate-7-phosphate synthase (aroG) known to undergo negative feedback control by binding to phenylalanine may be modified so that the negative feedback control is reduced or modified so that the control is released. The gene encoding the protein involved in tryptophan biosynthesis may be one type of gene or two or more types of genes. In addition, the gene encoding the protein involved in secologanin biosynthesis may be one type of gene or two or more types of genes.

Among these, the gene encoding the protein involved in tryptophan biosynthesis may contain at least one gene selected from aroG, trpD, and trpE genes, contains a combination of trpD gene and trpE gene and/or aroG gene, or contains aroG, trpD, and trpE genes. In addition, the trpE gene may be a trpE gene modified to suppress feedback control of anthranilate synthase by tryptophan. The aroG gene may be an aroG gene modified to suppress feedback control of 3-deoxy-D-arabino-heptulosonate-7-phosphate synthase by phenylalanine.

Description of the aroG, trpD, and trpE Genes

aroG Gene

The aroG is a gene encoding an enzyme involved in the synthesis of the first precursor, 3-deoxy-D-arabino heptulosonate 7 phosphate synthase (NAHP), of aromatic amino acids during tryptophan biosynthesis, namely 3-deoxy-7-phosphoheptulonate synthase (AroG). The action of the enzyme synthesizes NAHP from phosphoenolpyruvic acid and D-erythrose 4-phosphate, and furthermore, NAHP is metabolized into chorismic acid through several metabolic processes, which ultimately increases the amount of tryptophan biosynthesis. The majority of plants and microorganisms are applicable as organisms having a gene (aroG) encoding the enzyme, and examples thereof include E. coli, Corynebacterium glutamicum, Bacillus subtilis, Arabidopsis thaliana, Oryza sativa, and the like. In the present disclosure, the aroG gene may be a plant-derived and/or microorganism-derived aroG gene. Preferably, the aroG gene is a gene derived from Escherichia coli (E. coli).

In the present disclosure, the aroG gene may have a mutation provided that the protein obtained by expressing the gene functions as 3-deoxy-7-phosphoheptulonate synthase. The aroG gene may have 90% or more homology with the aroG gene possessed by any of the above plants or microorganisms (preferably E. coli), 95% or more homology with the sequence, 98% or more homology with the sequence, 99% or more homology with the sequence, or 100% homology with the sequence.

The above mutant gene of aroG is preferably modified so that the feedback control by phenylalanine is released. By using the mutant gene, negative feedback control does not occur even when the amount of tryptophan biosynthesis increases, so that the amount of tryptophan biosynthesis can be further increased. As such aroG gene, aroG4 gene, aroG15 gene, and the like are known. For aroG4 gene, it is possible to refer to APPLIED AND ENVIRONMENTAL MICROBIOLOGY February 1997, p. 761-762. The same reference can be made also for aroG15 gene.

The aroG gene introduced into a plant in the present disclosure may be a gene having a DNA sequence having 90% or more homology with the DNA sequence set forth in SEQ ID NO: 26 or 27 derived from Escherichia coli, a gene having a DNA sequence having 95% or more homology with the sequence, a gene having a DNA sequence having 98% or more homology with the sequence, a gene having a DNA sequence having 99% or more homology with the sequence, a gene having a DNA sequence having 99.5% or more homology with the sequence, a gene having a DNA sequence having 99.9% or more homology with the sequence, a gene having the DNA sequence set forth in SEQ ID NO: 26 or 27, or a gene having the DNA sequence set forth in SEQ ID NO: 27 (aroG4).

trpD Gene

The trpD is a gene encoding anthranilate phosphoribosyl transferase (TrpD). The action of the enzyme synthesizes pyrophosphoric acid and anthranilic acid from anthranilic acid and 5-phosphoribosyl diphosphate, and the synthesized anthranilic acid is metabolized through several metabolic processes, which ultimately increases the amount of tryptophan biosynthesis. The majority of plants and microorganisms are applicable as organisms having the gene, and examples thereof include E. coli, Corynebacterium glutamicum, Bacillus subtilis, Arabidopsis thaliana, Oryza sativa, and the like. In the present disclosure, the trpD gene may be a plant- and/or microorganism-derived trpD gene. Preferably, the trpD gene is a gene derived from Escherichia coli (E. coli).

In the present disclosure, the trpD gene may have a mutation provided that the protein obtained by expressing the gene functions as anthranilate phosphoribosyl transferase. The trpD gene may have 90% or more homology with the trpD gene possessed by any of the above plants or microorganisms (preferably E. coli), 95% or more homology with the sequence, 98% or more homology with the sequence, 99% or more homology with the sequence, or 100% homology with the sequence.

The trpD gene introduced into a plant of the present disclosure may be a gene having a DNA sequence having 90% or more homology with the DNA sequence set forth in SEQ ID NO: 28 derived from Escherichia coli, a gene having a DNA sequence having 95% or more homology with the sequence, a gene having a DNA sequence having 98% or more homology with the sequence, a gene having a DNA sequence having 99% or more homology with the sequence, a gene having a DNA sequence having 99.5% or more homology with the sequence, a gene having a DNA sequence having 99.9% or more homology with the sequence, or a gene having the DNA sequence set forth in SEQ ID NO: 28.

trpE Gene

The trpE gene is a gene encoding the a subunit (TrpE) of anthranilate synthase. The action of the enzyme synthesizes anthranilic acid, glutamic acid, and pyruvic acid from chorismic acid and glutamine, and the synthesized anthranilic acid is metabolized through several metabolic processes, which ultimately increases the amount of tryptophan biosynthesis. The majority of plants and microorganisms are applicable as organisms having the gene, and examples thereof include E. coli, Corynebacterium glutamicum, Bacillus subtilis, Arabidopsis thaliana, Oryza sativa, and the like. In the present invention, the trpE gene may be a plant- and/or microorganism-derived trpE gene. Preferably, the trpE gene is a gene derived from Escherichia coli (E. coli).

In the present disclosure, the trpE gene may have a mutation provided that the protein obtained by expressing the gene functions as the α subunit of anthranilate synthase and acts as anthranilate synthase together with the β subunit. The trpE gene may have 90% or more homology with the trpE gene possessed by any of the above plants or microorganisms (preferably E. coli), 95% or more homology with the sequence, 98% or more homology with the sequence, 99% or more homology with the sequence, or 100% homology with the sequence.

The above mutant gene of trpE is preferably modified to suppress the feedback control of anthranilate synthase by tryptophan. As such trpE gene, trpE8 gene, TRP4 gene, and the like are known. For trpE8 gene, it is possible to refer to WO 94/08031 and Published Japanese Translation of PCT International Application No. Hei 7-507693. For TRP4 gene, it is possible to refer to THE PLANT CELL 1993 VOL 5, p 1011-1027.

The trpE gene introduced into a plant of the present disclosure may be a gene having a DNA sequence having 90% or more homology with the DNA sequence set forth in SEQ ID NO: 29 or 30 derived from Escherichia coli, a gene having a DNA sequence having 95% or more homology with the sequence, a gene having a DNA sequence having 98% or more homology with the sequence, a gene having a DNA sequence having 99% or more homology with the sequence, a gene having a DNA sequence having 99.5% or more homology with the sequence, a gene having a DNA sequence having 99.9% or more homology with the sequence, a gene having the DNA sequence set forth in SEQ ID NO: 29 or 30, or a gene having the DNA sequence set forth in SEQ ID NO: 30 (trpE8).

Note that, in the present disclosure, the protein involved in tryptophan biosynthesis may have a signal sequence for transfer of aldolase derived from Catharanthus roseus to chloroplast. The signal sequence is preferably linked to the N-terminal side of the protein involved in tryptophan biosynthesis. In addition, the DNA sequence encoding the signal sequence is preferably linked to the 5′ terminal side of each gene encoding the protein involved in tryptophan biosynthesis. Having the signal sequence facilitates transport of the expressed protein into chloroplast. Since tryptophan is synthesized in chloroplast, having the signal sequence can increase the amount of tryptophan synthesis.

In addition, the DNA sequence encoding the signal sequence may be linked to the 5′ terminal side of each gene encoding the protein involved in tryptophan biosynthesis and have no stop codon. The absence of a stop codon allows the signal sequence and the protein involved in tryptophan biosynthesis to be expressed as a single protein, and therefore the protein involved in tryptophan biosynthesis is transferred to chloroplast through the N-terminal signal sequence.

5. Gene Encoding Protein Involved in Secologanin Biosynthesis

Examples of the gene encoding the protein involved in secologanin biosynthesis include the ORCA4 gene, the gene encoding secologanin synthase, the gene encoding loganate methyltransferase, the gene encoding iridoid synthase, and the gene encoding geraniol-10-dehydrogenase. Among these, the ORCA4 gene is preferable. The gene encoding the protein involved in secologanin biosynthesis may be one type of gene or two or more types of genes.

ORCA4 Gene

ORCA (octadecanoid derivative-Responsive Catharanthus AP2-domain) 4 gene is one of the ORCA gene clusters, is a gene presumed to have a master regulator function of an enzyme gene involved in the synthesis of Catharanthus roseus isoprenoid, and is known to be involved in secologanin biosynthesis. When the gene is expressed, the synthesis amount of secologanin synthase (SLS) and loganate methyltransferase (LAMT) increases (Non Patent Literature 2).

The ORCA4 gene may be a gene derived from Catharanthus roseus. The ORCA4 gene derived from Catharanthus roseus may be a gene having a DNA sequence having 90% or more homology with the DNA sequence set forth in SEQ ID NO: 32, a gene having a DNA sequence having 95% or more homology with the sequence, a gene having a DNA sequence having 98% or more homology with the sequence, a gene having a DNA sequence having 99% or more homology with the sequence, a gene having a DNA sequence having 99.5% or more homology with the sequence, a gene having a DNA sequence having 99.9% or more homology with the sequence, or a gene having the DNA sequence set forth in SEQ ID NO: 32.

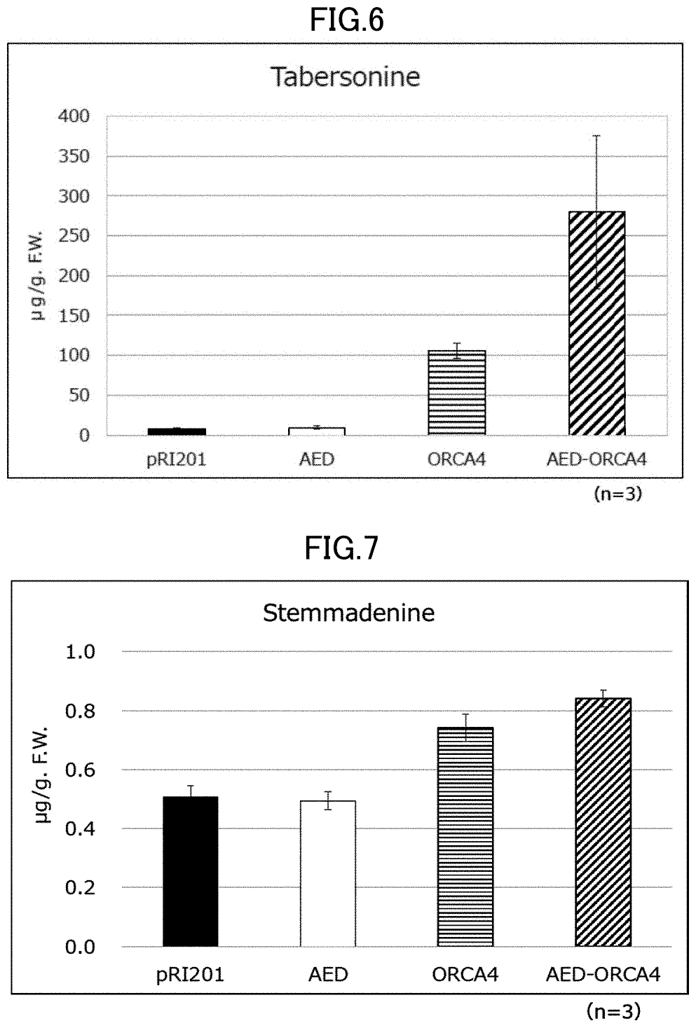

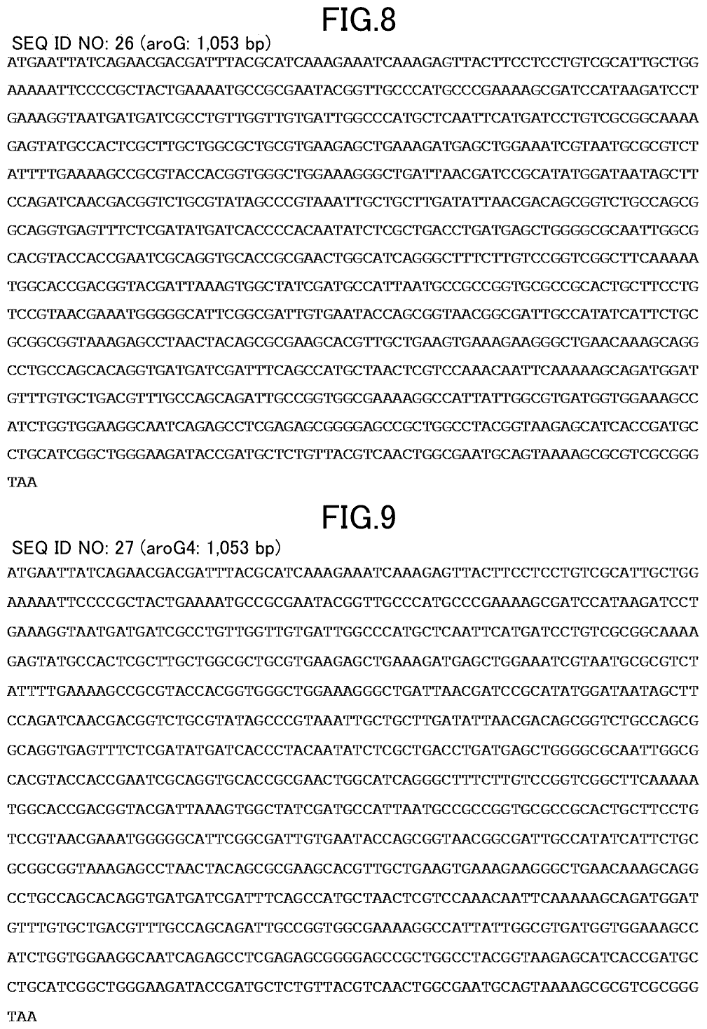

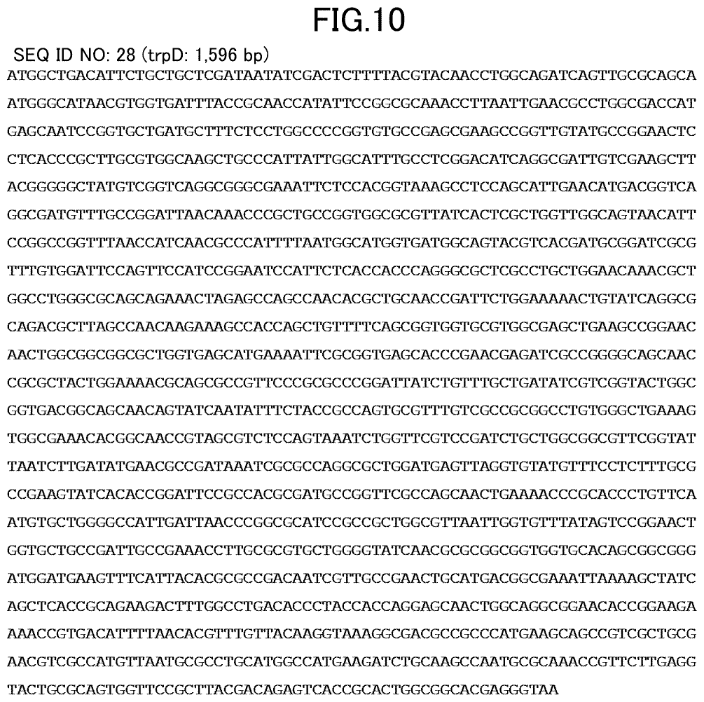

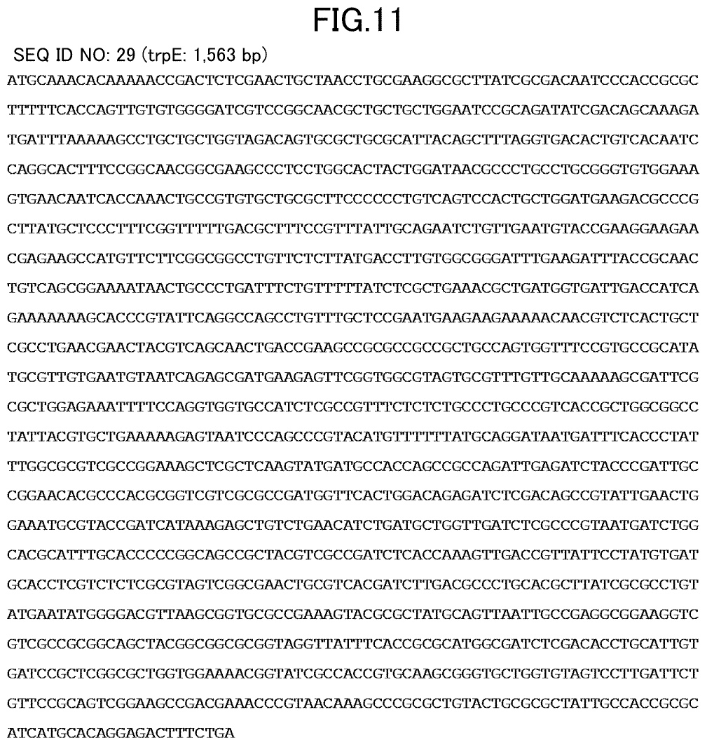

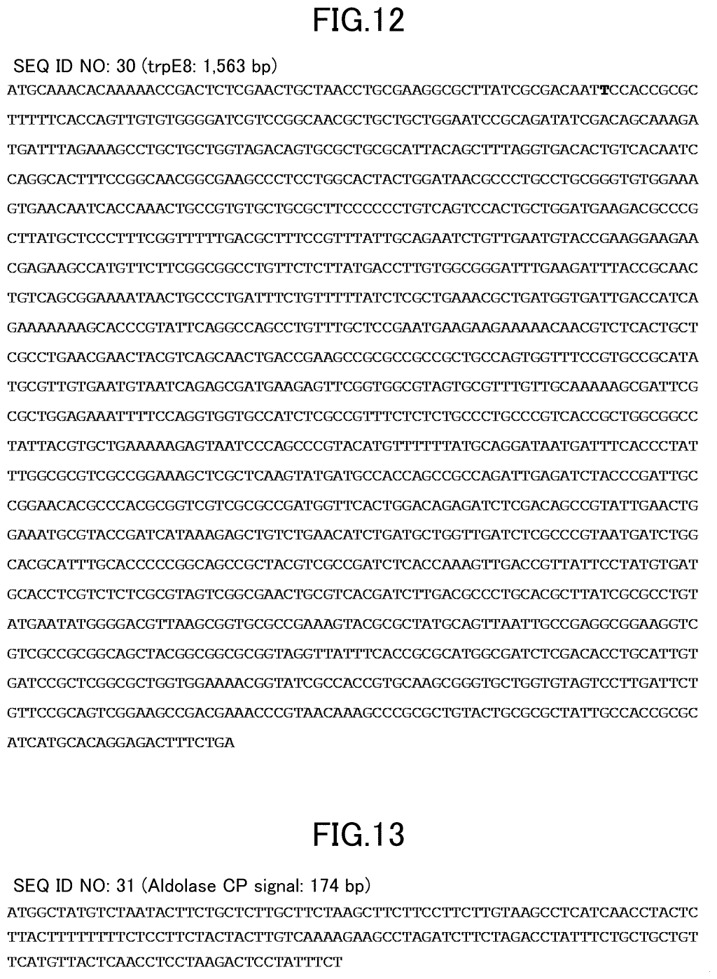

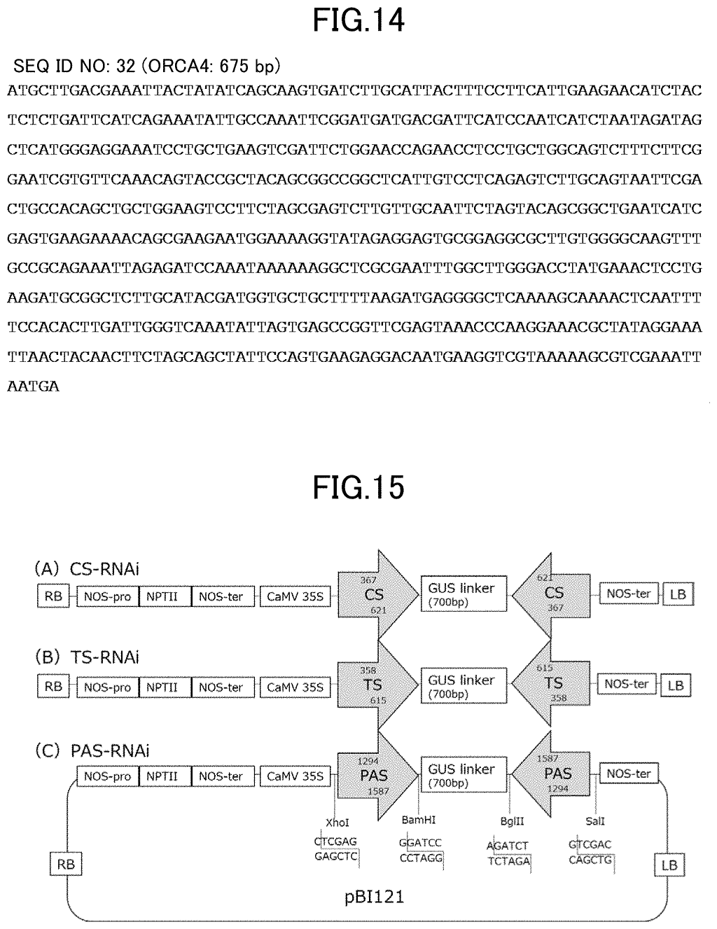

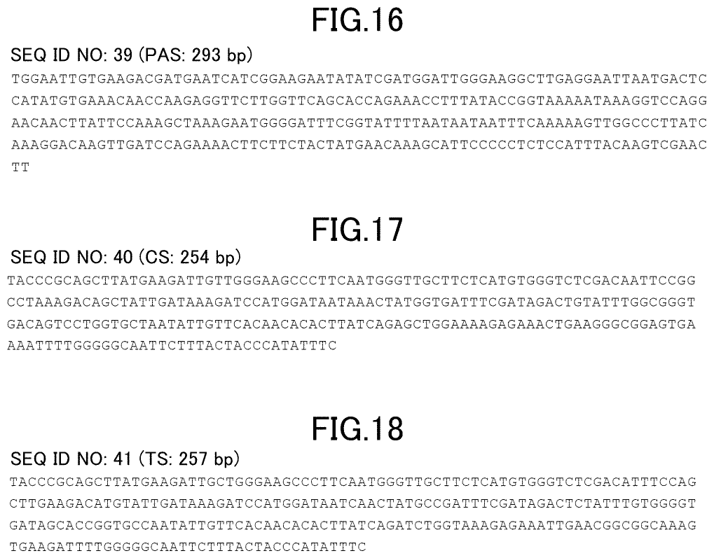

In the present disclosure, a protein involved in tryptophan biosynthesis (or a polypeptide) and a protein involved in secologanin biosynthesis (or a polypeptide) are expressed, using genetic engineering, in a plant of the genus Catharanthus in the family Apocynaceae, preferably the proteins are expressed in Catharanthus roseus by introducing a gene encoding the protein involved in tryptophan biosynthesis and a gene encoding the protein involved in secologanin biosynthesis into the plant, thereby making it possible to significantly increase the production amount of alkaloids such as tabersonine. It is not clear why expression of these proteins can significantly increase the amount of alkaloids produced. Non Patent Literature 1 states that the expression of a mutant trpE gene encoding anthranilate synthase with attenuated feedback inhibition by tryptophan increases the tryptophan content in hairy roots. Therefore, the present inventors considered that the amount of alkaloids might be increased by increasing the amounts of tryptophan and tryptamine also in the methods of the present disclosure. However, when a gene that enhances tryptophan-producing ability was introduced into Catharanthus roseus, the amount of tryptophan increased, but the amount of alkaloid did not increase at all (see the AED groups in FIGS. 5 and 6). On the other hand, in the methods of the present disclosure, when a gene encoding the protein involved in tryptophan biosynthesis and a gene encoding the protein involved in secologanin biosynthesis were introduced into Catharanthus roseus, the amount of tabersonine produced was significantly increased unexpectedly from the introduction of a gene encoding the protein involved in tryptophan biosynthesis (see AED-ORCA4 in FIGS. 5 and 6). As above, in the methods of the present disclosure, it is not clear why the amount of alkaloids significantly increases by introducing a gene encoding the protein involved in tryptophan biosynthesis and a gene encoding the protein involved in secologanin biosynthesis into Catharanthus roseus, as compared with the case where only the gene encoding the protein involved in tryptophan biosynthesis is introduced into the plant, which is an unexpected result.

6. Method of Introducing Gene

The method of introducing a gene encoding a protein involved in tryptophan biosynthesis and a gene encoding a protein involved in secologanin biosynthesis into a plant is not particularly limited as long as the gene is introduced into the plant, and it is preferable to introduce a gene encoding the protein involved in tryptophan biosynthesis and a gene encoding the protein involved in secologanin biosynthesis into a plant of the genus Catharanthus in the family Apocynaceae using a vector containing the gene encoding the protein involved in tryptophan biosynthesis and the gene encoding the protein involved in secologanin biosynthesis.

In addition, the methods of the present disclosure may introduce into the plant the gene encoding the protein involved in tryptophan biosynthesis and the gene encoding the protein involved in secologanin biosynthesis such that the gene encoding the protein involved in tryptophan biosynthesis and the protein involved in secologanin biosynthesis are transiently expressed in the plant.

In the present disclosure, the term “transiently expressed” means that the protein encoding the gene is transiently expressed by introducing the gene, and is used to distinguish from a stable expression system of a transgene by a transformant.

Examples of the method of introducing a gene encoding a protein involved in tryptophan biosynthesis and a gene encoding a protein involved in secologanin biosynthesis into a plant of the genus Catharanthus in the family Apocynaceae include the agroinfiltration method and the agroinoculation method. By these methods, proteins are transiently expressed in plants.

The agroinfiltration method is a method of immersing a plant in a suspension of Agrobacterium having a target gene and depressurizing and releasing the depressurization to allow the Agrobacterium suspension to penetrate into the plant.

The agroinoculation method is a method in which an Agrobacterium suspension having a target gene is directly injected into a plant tissue with pressurization, using a syringe or the like from which an injection needle has been removed.

Among these, in the methods of the present disclosure, it is preferable to introduce a gene encoding the protein involved in tryptophan biosynthesis and a gene encoding the protein involved in tryptophan biosynthesis into a plant of the genus Catharanthus in the family Apocynaceae by the agroinfiltration method.

In the infiltration method, a plant is, for example, immersed in a suspension prepared by adding Agrobacterium, containing a vector containing a gene encoding a protein involved in tryptophan biosynthesis and a gene encoding a protein involved in secologanin biosynthesis, or a mixture of vectors containing at least one gene selected from the gene encoding the protein involved in tryptophan biosynthesis and the gene encoding the protein involved in secologanin biosynthesis, the vector mixture containing the gene encoding the protein involved in tryptophan biosynthesis and the gene encoding the protein involved in secologanin biosynthesis in the entire mixture, to water or 2-morpholinoethanesulfonic acid (MES) buffer in an amount such that the OD600 value is about 0.5 to 2.0, and is exposed to a reduced pressure of about −0.07 to −0.09 MPa for 2 to 5 minutes, thereby making it possible to introduce the genes into the plant.

The vector that can be used in the methods of the present disclosure is not particularly limited as long as it can express the above genes in a plant, and various plasmid vectors can be used. For example, pRI201-AN vector (Takara Bio), pBI vector (Takara Bio), pRI vector (Takara Bio), pFAST vector (Thermo Fisher Scientific), and pSuperAgro vector (Inplanta Innovations) can be used. Among these, the pRI201-AN vector is preferable.

Examples of the method of incorporating a gene encoding a protein involved in tryptophan biosynthesis and a gene encoding a protein involved in secologanin biosynthesis into a vector include a method of incorporating by cleaving with a restriction enzyme and connecting by a ligation reaction, and a method of incorporating by a PCR reaction such as In-fusion method or Gibson assembly method.

7. Extraction

The methods of the present disclosure may include a step of expressing the protein in the plant and then extracting an alkaloid from the plant. Examples of the method of extracting alkaloids from plants include a method of crushing fresh leaves of a plant and extracting with a solvent such as methanol, a method of crushing leaves of a dried plant and extracting with a solvent such as methanol, and the like.

In the method of extraction from fresh leaves, for example, alkaloids can be extracted from plants by freezing with liquid nitrogen or the like, crushing, adding 99% methanol, and shaking at 30° C. for about 2 hours.

In the extraction method from dried leaves, for example, alkaloids can be extracted from plants by crushing freeze-dried leaves with a mixer or the like, adding 99% methanol, and shaking at 30° C. for about 2 hours.

8. Plant

The plant used in the methods of the present disclosure is a plant of the genus Catharanthus in the family Apocynaceae. Among these, Catharanthus roseus is more preferable.

In addition, in the methods of the present disclosure a protein involved in tryptophan biosynthesis (or a polypeptide) and a protein involved in secologanin biosynthesis (or a polypeptide) are preferably expressed, using genetic engineering, in the aerial part of a plant of the genus Catharanthus in the family Apocynaceae, and may be expressed in the leaves and/or stems of the plant.

In addition, the methods of the present disclosure may introduce a gene encoding the protein involved in tryptophan biosynthesis and a gene encoding the protein involved in secologanin biosynthesis into the aerial part of the plant, and may introduce them into the leaves and/or stems of the plant.

9. Alkaloid>

The methods of the present disclosure may be capable of increasing the amount of alkaloids produced in plants. Therefore, alkaloids can be produced with high efficiency.

The alkaloids produced using the methods of the present disclosure may be at least one selected from vinca alkaloids (particularly vinblastine and vincristine) and biosynthetic intermediates thereof, at least one selected from the group consisting of tabersonine, vindoline, catharanthine, vinblastine, vincristine, ajmalicine, and serpentine, at least one selected from the group consisting of tabersonine, stemmadenine, and strictosidine, at least one selected from the group consisting of tabersonine and stemmadenine, or tabersonine.

The structures of tabersonine, vindoline, catharanthine, vinblastine, vincristine, ajmalicine, serpentine, stemmadenine, and strictosidine are as follows.

| | |
|---|---|
| (Tabersonine) | (Vindoline) |
| 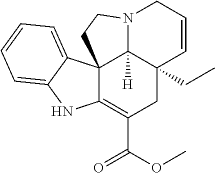 | 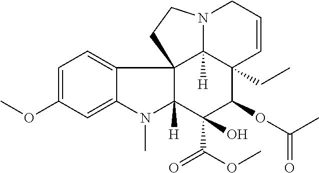 |
| (Catharanthine) | (Vinblastine) |
| 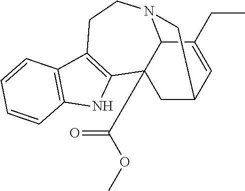 [SMILES: CCC1=CC2CN3CCc4c([nH]c5ccccc45)C(C(=O)OC)(C2)C13] | 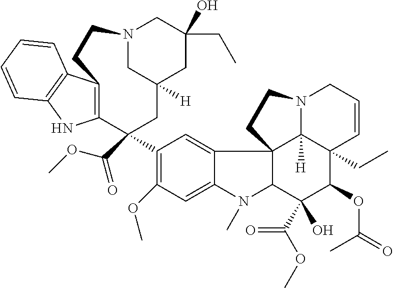 |
| (Vincristine) | (Ajmalicine) |
| 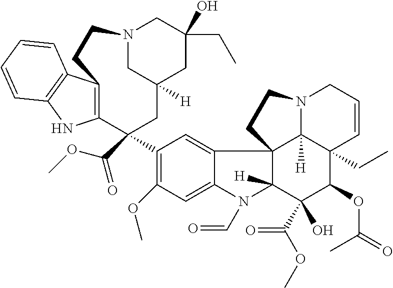 | 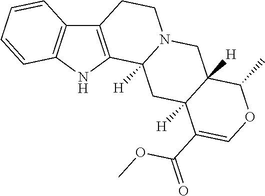 [SMILES: COC(=O)C1=CO[C@@H](C)[C@H]2CN3CCc4c([nH]c5ccccc45)[C@@H]3C[C@H]12] |
| (Serpentine) | (Strictosidine) |
| 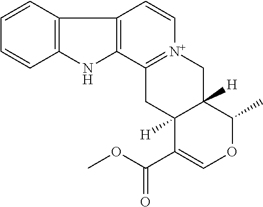 [SMILES: COC(=O)C1=CO[C@@H](C)[C@H]2C[n+]3ccc4c([nH]c5ccccc54)c3C[C@H]12] | 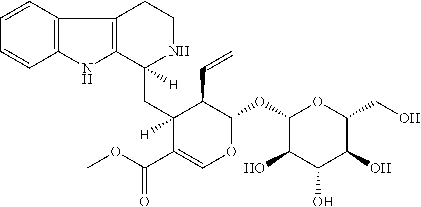 [SMILES: C=C[C@H]1[C@H](O[C@@H]2O[C@H](CO)[C@@H](O)[C@H](O)[C@H]2O)OC=C(C(=O)OC)[C@H]1C[C@@H]1NCCc2c1[nH]c1ccccc21] |
| (Stemmadenine) | |
| 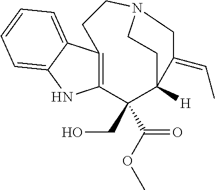 [SMILES: C/C=C1/CN2CCc3c([nH]c4ccccc34)[C@@](CO)(C(=O)OC)[C@@H]1CC2] | |

Vinca alkaloids (vinblastine, vincristine, vindesine, and vinorelbine) are known to exert potent microtubule polymerization inhibitory activity. They are also known to exert an anticancer agent action against Hodgkin's disease, malignant lymphoma, and the like.

Therefore, the methods of the present disclosure may also be useful as a method of producing an alkaloid or an alkaloid-containing composition for use in these applications.

In addition, the methods of the present disclosure may further include a step of controlling the biosynthesis of a desired alkaloid or of different alkaloids existing upstream and/or downstream of the desired alkaloid in the alkaloid biosynthetic pathway. This can further increase the content of the desired alkaloid contained in the plant. Examples of the method of controlling the biosynthesis of a desired alkaloid or of different alkaloids existing upstream and/or downstream of the desired alkaloid include a method of suppressing the biosynthesis of different alkaloids existing downstream of the desired alkaloid, and a method of promoting the biosynthesis of the desired alkaloid or different alkaloids existing upstream thereof.

The method of suppressing the biosynthesis of different alkaloids existing downstream of the desired alkaloid includes a method of suppressing the expression of mRNA level of a gene involved in the biosynthesis of the different alkaloid by, for example, introducing into the plant an siRNA or a RNAi expression vector for a gene involved in the biosynthesis of the different alkaloid. This suppresses the biosynthesis of different alkaloids existing downstream of the desired alkaloid and further increases the content of the desired alkaloid in the plant.

The method of promoting the biosynthesis of the desired alkaloid or different alkaloids existing upstream thereof may include a method of promoting the biosynthesis of the desired alkaloid or the different alkaloids existing upstream thereof by, for example, introducing into a plant a vector containing a gene encoding a protein involved in the biosynthesis of the desired alkaloid or the different alkaloids existing upstream thereof. This increases the amount of the desired alkaloid biosynthesized and further increases the content of the desired alkaloid in the plant.

A second aspect of the present disclosure includes a vector containing a gene encoding a protein involved in tryptophan biosynthesis and a gene encoding a protein involved in secologanin biosynthesis. Examples of the gene encoding a protein involved in tryptophan biosynthesis, the gene encoding a protein involved in secologanin biosynthesis, and the vector include the above-mentioned ones.

The vector may further contain a DNA sequence encoding a signal sequence for transfer of aldolase derived from Catharanthus roseus to chloroplast. The DNA sequence encoding the aldolase signal sequence is preferably the DNA sequence having the sequence set forth in SEQ ID NO: 31. The DNA sequence encoding the signal sequence is preferably linked to the 5′ terminal side of each gene encoding a protein involved in tryptophan biosynthesis. Having the signal sequence facilitates transport to chloroplast of the protein obtained by expressing the above gene after introducing the vector into a plant. Since tryptophan is synthesized in chloroplast, having the signal sequence can increase the amount of tryptophan synthesis.

In addition, the DNA sequence encoding the signal sequence may be linked to the 5′ terminal side of each gene encoding the protein involved in tryptophan biosynthesis and have no stop codon. The absence of a stop codon allows the signal sequence and the protein involved in tryptophan biosynthesis to be expressed as a single protein, and therefore the protein involved in tryptophan biosynthesis is transferred to chloroplast through the N-terminal signal sequence.

A third aspect of the present disclosure includes Agrobacterium containing a vector containing a gene encoding a protein involved in tryptophan biosynthesis and a gene encoding a protein involved in secologanin biosynthesis, or a mixture of vectors containing at least one gene selected from the gene encoding the protein involved in tryptophan biosynthesis and the gene encoding the protein involved in secologanin biosynthesis, the vector mixture containing the gene encoding the protein involved in tryptophan biosynthesis and the gene encoding the protein involved in secologanin biosynthesis in the entire mixture. Examples of the gene encoding a protein involved in tryptophan biosynthesis, the gene encoding a protein involved in secologanin biosynthesis, and the vector include the above-mentioned ones.

Examples of the vector mixture include a vector mixture in which one of the vector mixtures has a gene encoding a protein involved in tryptophan biosynthesis and another one has a gene encoding a protein involved in secologanin biosynthesis. In addition, in the case of introducing multiple genes each of which encodes a protein involved in tryptophan biosynthesis, it may be a mixture including: a mixture of vectors having these genes; and a vector containing a gene encoding a protein involved in secologanin biosynthesis.

The vector of the second aspect or the Agrobacterium of the third aspect is suitable for use in the method of the first aspect of the disclosure.

A fourth aspect of the present disclosure includes a plant of the genus Catharanthus in the family Apocynaceae containing a vector containing a gene encoding a protein involved in tryptophan biosynthesis and a gene encoding a protein involved in secologanin biosynthesis, or a mixture of vectors containing at least one gene selected from the gene encoding the protein involved in tryptophan biosynthesis and the gene encoding the protein involved in secologanin biosynthesis, the vector mixture containing the gene encoding the protein involved in tryptophan biosynthesis and the gene encoding the protein involved in secologanin biosynthesis in the entire mixture.

Examples of the gene encoding a protein involved in tryptophan biosynthesis, the gene encoding a protein involved in secologanin biosynthesis, the vector, and the plant include the above-mentioned ones.

The plant of the fourth aspect can be obtained by, for example, introducing a gene into the plant body, which may be the aerial part of the plant, or which may be the leaves and/or stems of the plant, using the vector of the second aspect or the Agrobacterium of the third aspect.

The plant of the fourth aspect (particularly the site in which the vector is introduced) may have a tabersonine production amount (the number of moles per unit mass of the fresh plant part) that is 5 times or more, 10 times or more, 50 times or more, 100 times or more, or 150 times or more as compared with a wild strain plant of the genus Catharanthus in the family Apocynaceae (particularly the same site as the above site).

A fifth aspect of the present disclosure includes a plant of the genus Catharanthus in the family Apocynaceae, which has been transformed so as to overproduce alkaloids by introducing a vector containing a gene encoding a protein involved in tryptophan biosynthesis and a gene encoding a protein involved in secologanin biosynthesis, or a mixture of vectors containing at least one gene selected from the gene encoding the protein involved in tryptophan biosynthesis and the gene encoding the protein involved in secologanin biosynthesis, the vector mixture containing the gene encoding the protein involved in tryptophan biosynthesis and the gene encoding the protein involved in secologanin biosynthesis in the entire mixture.

Examples of the gene encoding a protein involved in tryptophan biosynthesis, the gene encoding a protein involved in secologanin biosynthesis, the vector, the alkaloid, and the plant include the above-mentioned ones.

A plant of the fifth aspect can be obtained by, for example, introducing genes into the cells of the plant using the vector of the second aspect or the Agrobacterium of the third aspect. After gene introduction, stable expression strains having these genes incorporated in the genome of the plant cells may be selected. Examples of a method of selecting stable expression strains include a method in which a drug resistance gene is introduced into plant cells together with the above genes, and the plant cells are passaged in a drug-containing medium. A transformed plant can be obtained by differentiating and growing the obtained plant cells.

The term “overproduce” means that the production amount of alkaloids (the number of moles per unit mass of the plant or plant part), preferably the production amount of tabersonine, is increased by 1.5 times or more as compared with the production amount of the wild strain plant of the genus Catharanthus in the family Apocynaceae. The amount of alkaloids produced may be increased by 2 times or more, 5 times or more, 10 times or more, 50 times or more, 100 times or more, or 150 times or more.

### EXAMPLES

Hereinafter, the usefulness of the present disclosure is specifically described with reference to Examples. However, the present disclosure is not limited to these.

### Example 1

1. Materials and Methods

(1) Acquisition of Gene Encoding Target Protein and Addition of Signal Sequence

Each of the aroG gene, trpE gene, and trpD gene was acquired from E. coli DNA by PCR using primers (SEQ ID NOS: 1 to 6) based on the gene sequence information (aroG: SEQ ID NO: 26, trpE: SEQ ID NO: 29, trpD: SEQ ID NO: 28). All the genes obtained by cloning using the TA cloning kit were subjected to sequence analysis, and it was confirmed that there was no difference with the base sequence information. PrimeSTAR Mutagenesis Basal Kit (Takara Bio) was used to carry out the PCR method, and the 150th amino acid of the amino acid sequence of aroG gene was mutated from proline (codon sequence: cca) to lysine (codon sequence: cta) to obtain aroG4 gene (SEQ ID NO: 27). Similarly, PrimeSTAR Mutagenesis Basal Kit (Takara Bio) was used to carry out the PCR method, and the 21st amino acid of the amino acid sequence of trpE gene was mutated from proline (codon sequence: ccc) to serine (codon sequence: tcc) and the 50th amino acid was mutated from lysine (codon sequence: aaa) to glutamic acid (codon sequence: gaa) to obtain trpE8 gene (SEQ ID NO: 30). The mutated gene was also subjected to sequence analysis to confirm that base substitution had occurred.

The ORCA4 gene derived from Catharanthus roseus, which is a transcriptional regulator of the isoprenoid metabolism system, was employed as a gene encoding a protein involved in secologanin biosynthesis, and the ORCA4 gene was acquired by synthesis based on the gene sequence information. The DNA sequence of the synthesized ORCA4 gene is as set forth in SEQ ID NO: 32.

Since tryptophan is synthesized by chloroplasts in plants, in order to transport the protein synthesized in plants to chloroplasts, a signal sequence for transfer of aldolase derived from Catharanthus roseus to chloroplast (GenBank: GU723954, 174 bp from N-terminal, SEQ ID NO: 31) was acquired by synthesis and ligated by the PCR method to the N-terminal side of each gene of aroG4, trpE8, and trpD (referred to as Ald-aroG4, Ald-trpE8, Ald-trpD, respectively). The ligation of the signal sequence and aroG4 gene was performed as follows. In order to connect the signal sequence for the transfer of aldolase to chloroplast with aroG4, PCR was conducted using the signal sequence gene (SEQ ID NO: 31) and the aroG4 gene (SEQ ID NO: 27) as templates and using two primer pairs, one of which is a primer pair having a primer prepared for the 5′ side of aldolase gene (SEQ ID NO: 7) and a reverse sequence primer prepared for the linking region between aldolase gene and aroG4 gene (SEQ ID NO: 9), and the other of which is a primer pair having a forward sequence primer prepared for the linking region aldolase gene and aroG4 gene (SEQ ID NO: 8) and a reverse sequence primer prepared for the 3′ side of aroG4 gene (SEQ ID NO: 2). Next, it was confirmed by electrophoresis that each amplified gene fragment had a target size, and then DNA was cut out from the gel and purified using Wizard SV Gel and PCR Clean-Up System (Promega). PCR was conducted using a mixture of the genes as a template and using a primer for hybridizing the 5′ side of aldolase gene (SEQ ID NO: 7) and a reverse sequence primer for hybridizing the 3′ side of aroG4 gene (SEQ ID NO: 2). Thereby, the genes were linked to each other. By the same method, the above-mentioned signal sequence and trpE8 gene were linked to each other using the primers of SEQ ID NOS: 4, 7, 10, and 11. The signal sequence and the trpD gene were linked to each other using the primers of SEQ ID NOS: 6, 7, 12, and 13.

(2) Creating Binary Vector for Expression

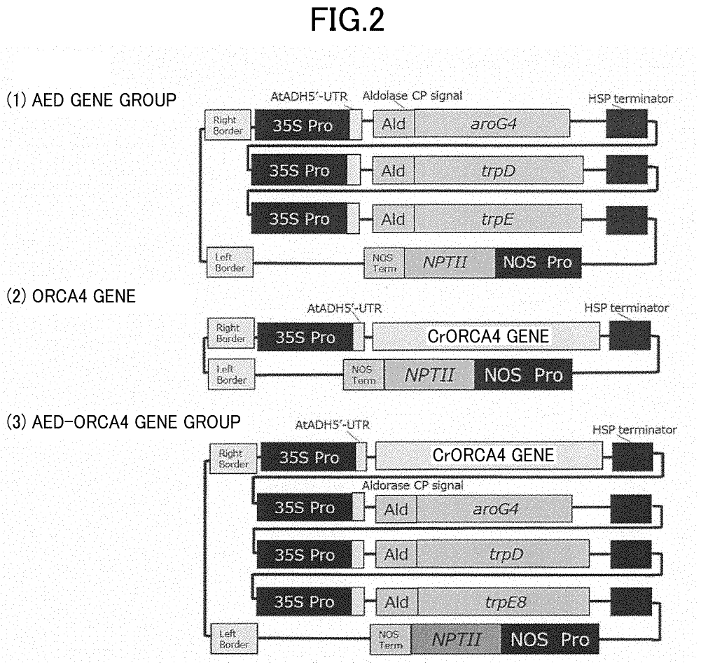

Each of the four genes thus obtained (Ald-aroG4, Ald-trpE8, Ald-trpD, ORCA4) was inserted between the promoter and terminator of the binary vector, pRI201-AN (TAKARA BIO.) to obtain a respective gene set. The gene set was amplified by the PCR method using the primers of SEQ ID NOS: 14 and 15 from the upstream of the promoter to the downstream of the terminator. Gibson Assembly System (NEW ENGLAND BioLabs) was used to ligate the gene set to complete three types of expression vectors (AED gene group expression vector, ORCA4 gene expression vector, AED-ORCA4 gene group expression vector). The ligated binary vector was transformed into Agrobacterium (Agrobacterium tumefaciens, LBA4404) and used for a transient expression experiment. The structure of the ligated vector is as shown in FIG. 2.

Note that, in the above description, the AED gene group expression vector is an expression vector of the gene group containing the aroG, trpE8, and trpD genes in the vector. The AED-ORCA4 gene group expression vector is an expression vector of the gene group containing aroG, trpE8, trpD, and ORCA4 genes in the vector. In FIG. 2, CrORCA4 means the ORCA4 gene derived from Catharanthus roseus, NPTII means the neomycin phosphotransferase II gene, 35S pro means the cauliflower mosaic virus 35S promoter, and NOS Pro means the nopaline synthase gene promoter.

(3) Transient Expression Treatment and Sampling

(3-a) Preparation of Agrobacterium Suspension

50 mg/l kanamycin was added as an antibiotic to 5 ml of YEP medium (1% yeast extract, 1% peptone, 0.5% sodium chloride), and Agrobacterium containing the target gene was inoculated with the medium, followed by shake-culturing overnight at 28° C. Moreover, on the next day, an antibiotic was added to 100 ml of YEP medium in the same manner, and the culture amplified in 5 ml of YEP medium was transplanted thereto, followed by culture overnight at 28° C. The culture was placed in a 50 ml Falcon tube and centrifuged at 4,600 rpm and 22° C. for 10 minutes to collect the Agrobacterium. The Agrobacterium were suspended in MES buffer (1.952 g/1 MES, 2.033 g/1 MgCl2.6H2O, pH 5.7), and 30 mg/l acetosyringone (3′5′-dimethoxy-4′-hydroxyacetophenone) was added thereto. The Agrobacterium suspension was adjusted so that the OD600 was 1.5, and was used for transient expression treatment by agroinfiltration using a 200 ml beaker.

(3-b) Cultivation of Catharanthus roseus

For the seeds of Catharanthus roseus, Pacifica XP from (TAKII & Co., Lid) was used. After seeding in Jiffy Seven™ and growing for about 2 weeks, they were transplanted to a 6 cm pot. They were further cultivated for about 3 weeks, and plants which had 6 to 7 true leaves were used as a material. Cultivation was carried out under the conditions of 16 hours photoperiod and 23° C.

(3-c) Transient Expression Treatment by Agroinfiltration and Sampling

For the seedling of Catharanthus roseus prepared in (3-b), the pot and soil portions were covered with aluminum foil. Then, the seedling was turned upside down and slowly placed in the Agrobacterium suspension placed in a beaker so that the whole aerial part was dipped in the suspension. The beaker was then placed in a desiccator, the pressure was reduced to −0.09 MPa using a vacuum pump, the condition was kept to stand for 2 minutes, and then the cock was opened to release the reduced pressure (the Agrobacterium suspension enters the epidermis of the leaf tissue). After the infection treatment, the leaves were left to stand indoors until they were dry, and then cultivated in a BioTRON room (16 hours photoperiod, 23° C.).

For gene expression analysis, amino acid analysis, and alkaloid analysis, about 100 to 200 mg of leaf tissues on day 7 after infection was cut out and sampled and stored at −80° C.

(4) Real-Time PCR Analysis

Total RNA was extracted from about 100 mg of leaf tissues using RNeasy Plant Mini Kit manufactured by QIAGEN. After performing DNase (RNase-Free DNase Set manufactured by QIAGEN) treatment to prevent the contamination of DNA, cDNA was created using Prime Script RT reagent kit II (TaKaRa Bio). Fast SYBR Green Master Mix was reacted, and RNA transcription amount was measured using 7500 Fast Real Time PCR System (Thermo Fisher Scientific Inc.).

(5) Analysis of Tryptophan Content

Leaf tissues in an amount of 200 mg were frozen in liquid nitrogen, crushed using MM300 (Retsch), and then 500 μl of 80% ethanol was added thereto. After shaking at room temperature for 30 minutes, centrifugation was performed at room temperature at 12,000 rpm for 20 minutes, and the supernatant was transferred to a new tube. 500 μl of 80% ethanol was added to the remaining precipitate, shaken at room temperature for 30 minutes, and then centrifuged at 12,000 rpm for 20 minutes at room temperature. The supernatant was transferred to the previous tube. This operation was repeated once more, and extraction was performed 3 times. The extract was adjusted to 1.5 ml and mixed well, and 600 μl of the extract was transferred to another tube. These were dried using a vacuum evaporator, and 600 μl of sterilized water was added and dissolved. Moreover, in order to remove proteins, 200 μl of chloroform was added, mixed well, and then centrifuged at 12,000 rpm for 10 minutes at room temperature. The supernatant was transferred to another new tube. To 180 μl of the purified solution, 20 μl of 0.2 N hydrochloric acid was added, mixed well, and then filtered using an Ultra-free-MC 0.45 filter. The filtrate was used to analyze the tryptophan content with a HITACHI high-speed amino acid analyzer L8800.

(6) Alkaloid Analysis

Leaf tissues in an amount of 200 mg were frozen in liquid nitrogen, crushed using MM300, and then added with 500 μl of methanol. After shaking at 30° C. for 2 hours, centrifugation was performed at room temperature and 12,000 rpm for 20 minutes. The supernatant was filtered using an Ultra-free-MC 0.45 filter, and the filtrate was used as a sample.

The measurement of tabersonine was performed using HPLC. For HPLC, a Capcell pac C18 UG120 (4.6×250 mm, 5 μm, Shiseido Company, Limited) column was used, followed by development using a mixture liquid of 30% (v/v) methanol and 0.1 M phosphoric acid (pH 2) at a flow rate of 1 ml/min for 80 minutes. The column temperature was 40° C., and detection was performed by UV and fluorescence.

The measurement of stemmadenine was performed using LC-MS. The LC apparatus used was Nexera X2 (Shimadzu), and the analysis was performed under the conditions of mobile phase A: 10 mM ammonium acetate/0.1% acetic acid/water, mobile phase B: methanol, flow rate: 0.3 mL/min, column: Waters Acquit HPLC BEH C18 (2.1×50 mm, 1.7 μm), column temperature: 40° C., gradient conditions: 0-15 min: 10% B-95% B, 15-16 min: 95% B, 17-20 min: 10% B. The standard (DMSO solution) and the Catharanthus roseus leaf extract (MeOH solution) were diluted with 60% methanol and analyzed. UV was detected at 190-400 nm. The MS apparatus used was Q-Exactive (Thermo), and FM scan measurement (molecule scan), PRM measurement (target molecule MS/MS), and DDA measurement (MS/MS data was acquired by automatic selection of down to the fifth intensity subjected to non-target MS/MS detection) were performed.

2. Results

(1) Results of Real-Time PCR Analysis

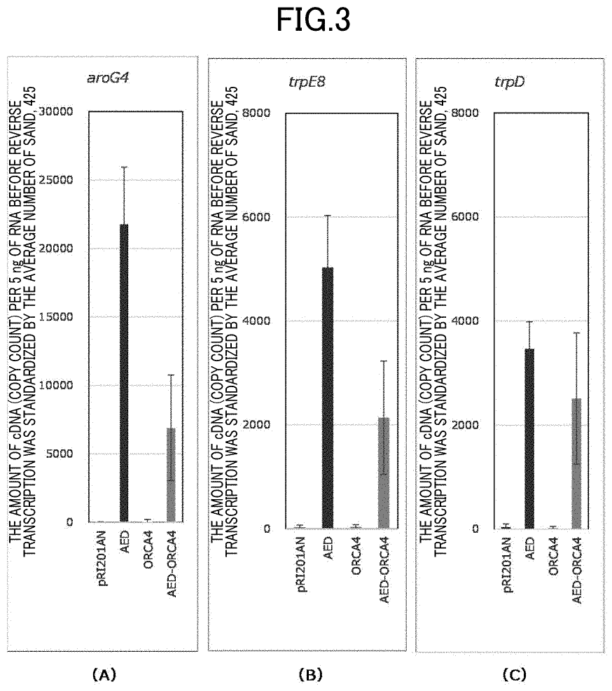

Real-Time PCR analysis was performed in order to confirm the gene expression at the mRNA level of the gene introduced by transient expression. The primers of SEQ ID NOS: 16 and 17 were used for the expression analysis of aroG4 gene, ones of SEQ ID NOS: 18 and 19 were used for the expression of trpE8 gene, and ones of SEQ ID NOS: 20 and 21 were used for the expression of trpD gene. As a result, the expression of aroG4, trpE8, and trpD genes was confirmed in the group infected with Agrobacterium containing the AED gene group expression vector (AED group) and the group infected with Agrobacterium containing the AED-ORCA4 gene group expression vector (AED-ORCA4 group). On the other hand, in the vector control (pRI201-AN) group not containing these genes and the group infected with Agrobacterium containing the ORCA4 gene vector (ORCA4 group), expression of these genes could not be confirmed (FIG. 3).

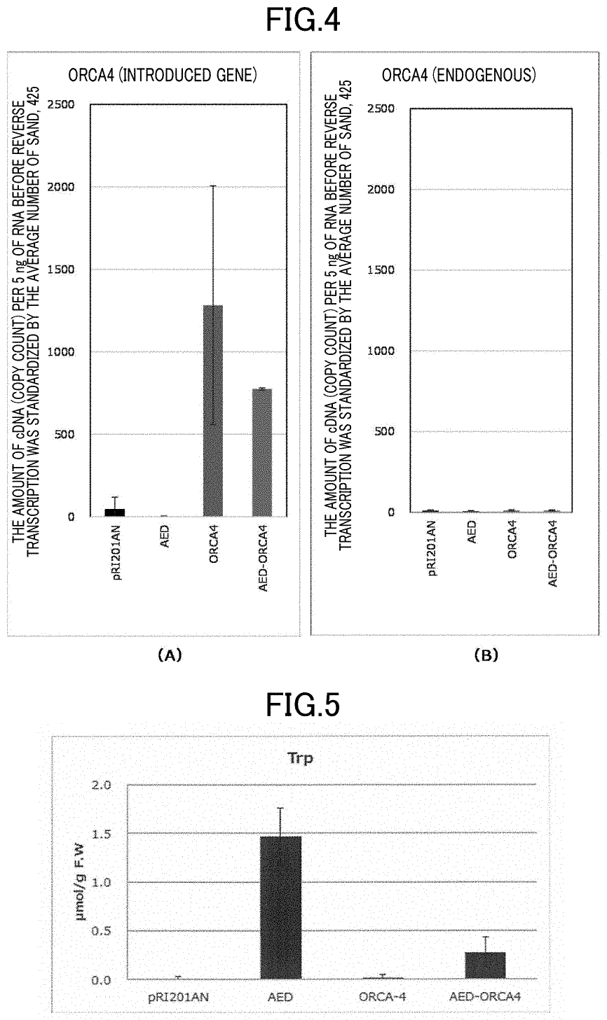

In addition, the expression of the introduced ORCA4 gene was confirmed using the primers of SEQ ID NOS: 22 and 23. As a result, transcriptional expression of the same gene was confirmed in ORCA4 group and AED-ORCA4 group. However, the expression of these genes could not be confirmed in the vector control (pRI201-AN) group and the AED group not containing the ORCA4 gene. Note that the expression of endogenous ORCA4 gene was also analyzed using the primers of SEQ ID NOS: 24 and 25 prepared in the endogenous ORCA4 gene-specific base sequence portion. As a result, it was found that the expression level of the endogenous ORCA4 gene was low (FIG. 4).

(2) Results of Analysis of Tryptophan Content

As a result of analysis of the tryptophan content, the tryptophan content in the pRI201-AN (vector control) group was 0.011 μmol/gF·W, whereas it was 1.471 μmol/gF·W. in the AED group and 0.280 μmol/g·F·W. in the AED-ORCA4 group, showing that the tryptophan (Trp) content in the AED group and the AED-ORCA4 group was significantly increased as compared to the pRI201-AN (vector control) group. In addition, in ORCA4 group, it was 0.017 μmol/g·F·W, which was similar to that of the vector control (FIG. 5).

(3) Results of Alkaloid Analysis

As a result of alkaloid analysis, the content of tabersonine was 7.6 μg/gF·W. in the pRI201-AN (vector control) group and 9.2 μg/gF·W. in the AED group, whereas the ORCA4 group had 105.7 μg/gF·W. (13.9 times higher compared to the control), and the AED-ORCA4 group had 279.9 μg/gF·W. (36.8 times higher compared to the control) (FIG. 6).

In addition, the content of stemmadenine was 0.51 μg/gF·W. in the pRI 201 AN group and 0.49 μg/gF·W. in the AED group, whereas the ORCA4 group had 0.74 μg/gF·W. (1.46 times higher compared to the control) and the AED-ORCA4 group had 0.84 μg/gF·W. (1.65 times higher compared to the control) (FIG. 7).

### Example 2

1. Materials and Methods

(1) Preparation of RNAi Expression Vector

An RNAi vector is prepared for the purpose of suppressing the expression, at the mRNA level, of each of the genes of an enzyme that metabolizes stemmadenine and biosynthesizes precondylocarpine acetate (precondylocarpine acetate synthase, PAS), an enzyme that biosynthesizes tabersonine (tabersonine synthase, TS), and an enzyme that biosynthesizes catharanthine (catharanthine synthase, CS).

293 bp (SEQ ID NO: 39) between 1,294 to 1,587 bp of the base sequence of the PAS gene, 254 bp (SEQ ID NO: 40) between 367 bp and 621 bp of the CS gene, and 358 bp to 615 bp (SEQ ID NO: 41) of the TS gene are used for the preparation of the RNAi vectors, and the vector is prepared as illustrated in FIG. 15. These vectors are transformed into Agrobacterium, LBA4404, and used for transient expression.

(2) Transient Expression Treatment and Sampling

Agrobacterium containing the AED-ORCA4 gene group and Agrobacterium containing the PAS-RNAi vector are each adjusted to have an OD600 value of 1.0, and then mixed at a ratio of 1:1. Similarly, Agrobacterium containing the AED-ORCA4 gene group, Agrobacterium containing the CS-RNAi vector, and Agrobacterium containing the TS-RNAi vector are each adjusted to have an OD600 value of 1.5, and then mixed at a ratio of 1:1:1. As a control, the original vector pRI 201-AN is used, which is transformed into Agrobacterium LBA4404. Agrobacterium containing the AED-ORCA4 gene group is also used as a control for confirming an increase in tabersonine.

(3) Real-Time PCR Analysis

Total RNA is extracted from about 100 mg of leaf tissues using RNeasy Plant Mini Kit manufactured by QIAGEN. After performing DNase (RNase-Free DNase Set manufactured by QIAGEN) treatment to prevent the contamination of DNA, cDNA is created using Prime Script RT reagent kit II (TaKaRa Bio). Fast SYBR Green Master Mix is reacted, and RNA transcription amount is measured using 7500 Fast Real Time PCR System (Thermo Fisher Scientific Inc.). Real-Time PCR of the PAS gene is performed with the primer combination of SEQ ID NOS: 33 and 34, and similarly, that of the CS gene is performed with the primer combination of SEQ ID NOS: 35 and 36, and that of the TS gene is performed with the primer combination of SEQ ID NOS: 37 and 38.

(4) Measurement of Alkaloid Content

Leaf tissues in an amount of 200 mg is frozen in liquid nitrogen and crushed using MM300, and then 500 μl of methanol is added thereto. After shaking at 30° C. for 2 hours, centrifugation is performed at room temperature and 12,000 rpm for 20 minutes. The supernatant is filtered using an Ultra-free-MC 0.45 filter, and the filtrate is used as a sample.

The measurement of tabersonine is performed using HPLC. For HPLC, a Capcell pac C18 UG120 (4.6×250 mm, 5 μm, Shiseido Company, Limited) column is used, followed by development using a mixture liquid of 30% (v/v) methanol and 0.1 M phosphoric acid (pH 2) at a flow rate of 1 ml/min for 80 minutes. The column temperature is 40° C., and detection is performed by UV and fluorescence.

The measurement of stemmadenine is performed using LC-MS. The LC apparatus used is Nexera X2 (Shimadzu), and the analysis is performed under the conditions of mobile phase A: 10 mM ammonium acetate/0.1% acetic acid/water, mobile phase B: methanol, flow rate: 0.3 mL/min, column: Waters Acquit HPLC BEH C18 (2.1×50 mm, 1.7 μm), column temperature: 40° C., gradient conditions: 0-15 min: 10% B-95% B, 15-16 min: 95% B, 17-20 min: 10% B. The standard (DMSO solution) and the Catharanthus roseus leaf extract (MeOH solution) are diluted with 60% methanol and analyzed. UV is detected at 190-400 nm. The MS apparatus used is Q-Exactive (Thermo), and FM scan measurement (molecule scan), PRM measurement (target molecule MS/MS), and DDA measurement (MS/MS data is acquired by automatic selection of down to the fifth intensity subjected to non-target MS/MS detection) are performed.

2. Results

(1) Real-Time PCR Analysis

In AED-ORCA4 group, AED-ORCA4+PAS-RNAi group, and AED-ORCA4+CS-RNAi+TS-RNAi group, the expression of mRNA for aroG4, trpE8, trpD, and ORCA4 genes can be confirmed, whereas expression cannot be confirmed in vector control (pRI201-AN) group not containing these genes.

Moreover, in the AED-ORCA4+PAS-RNAi group, the suppression of the PAS gene mRNA expression is observed as compared with the vector control group, and in the AED-ORCA4+CS-RNAi+TS-RNAi group, it can be confirmed that the expression of mRNA of CS and TS genes is suppressed.

(2) Alkaloid Analysis

As a result of alkaloid analysis, in the AED-ORCA4 group, the tabersonine content is significantly increased as compared with the vector control group, and in the AED-ORCA4+PAS-RNAi group and the AED-ORCA4+CS-RNAi+TS-RNAi group, on the other hand, the tabersonine content slightly increases compared to the vector control group, but decreases compared to the AED-ORCA4 group, and instead the stemmadenine content increases significantly.

3. Consideration 1

In the AED group, the alkaloid content could not be increased although the tryptophan content was very high. On the other hand, in the AED-ORCA4 group in which the transcriptional regulator ORCA4 of the isoprenoid metabolism system was linked to the AED, the content of tabersonine was significantly increased. This result was unexpected from each of the functions of the AED group and the ORCA4 group.

4. Consideration 2

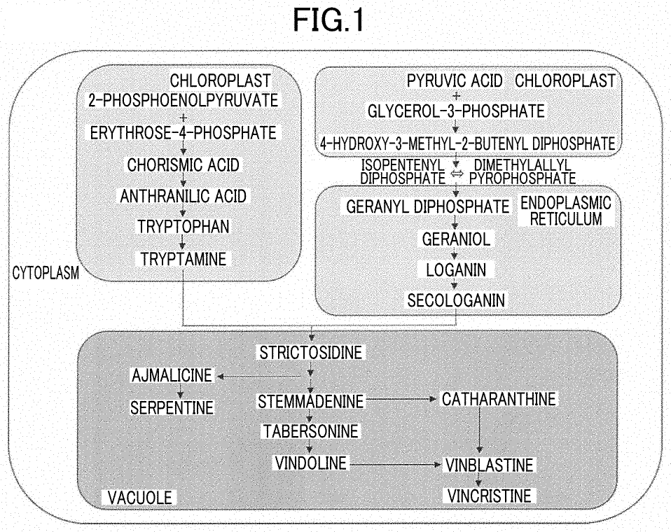

It is known that in plants of the genus Catharanthus in the family Apocynaceae such as Catharanthus roseus, tabersonine and vindoline are produced through the production of strictosidine and subsequent stemmadenine (FIG. 1). As a result of Example 1, the content of tabersonine was significantly increased, and the content of stemmadenine upstream thereof was also increased. From this, it is understood that the methods of the present disclosure increase the contents of various alkaloids existing upstream and downstream of the biosynthesis of tabersonine and stemmadenine. In addition, by controlling the expression of a gene involved in the biosynthesis of an alkaloid existing upstream and/or downstream of the desired alkaloid (suppressing the expression of a gene involved in the biosynthesis of an alkaloid existing downstream of the desired alkaloid, for example), it is believed that the amount of biosynthesis of the desired alkaloid can be further increased.

5. Consideration 3

It is known that in plants of the genus Catharanthus in the family Apocynaceae such as Catharanthus roseus, tabersonine and vindoline are produced through the production of strictosidine and subsequent stemmadenine (FIG. 1). It is understood that when the expression of mRNA of the gene encoding PAS, CS, TS, which are enzymes that metabolize stemmadenine of Example 2, is suppressed by the RNAi method, the stemmadenine content is increased.

TABLE 1
Primer Sequences Used to Construct Vector for Gene Expression.

| | | |
|---|---|---|
| SEQ ID | aroG4 FW: | 5′-ATG AAT TAT CAG AAC GAC GAT TTA-3′ |
| NO: 1 | | |
| SEQ ID | aroG4 RV: | 5′-TTA CCC GCG ACG CGC TTT TAC TGC-3′ |
| NO: 2 | | |
| SEQ ID | trpE8 FW: | 5′-ATG CAA ACA CAA AAA CCG ACT CTC-3′ |
| NO: 3 | | |
| SEQ ID | trpE8 RV: | 5′-TCA GAA AGT CTC CTG TGC ATG ATG-3′ |
| NO: 4 | | |
| SEQ ID | trpD FW: | 5′-ATG GCT GAC ATT CTG CTG CTC GAT AAT |
| NO: 5 | | ATC GAC TC-3′ |
| SEQ ID | trpD RV: | 5′-TTA CCC TCG TGC CGC CAG TGC GGT |
| NO: 6 | | GAC TCT GTC-3′ |
| SEQ ID | Aldolase-sp FW: | 5′-GGG CAT ATG GCT GAC ATT CTG CTG |
| NO: 7 | | CTC GAT AAT ATC GAC TC-3′ |
| SEQ ID | Ald-aroG4 FW2: | 5′-CCT AAG ACT CCT ATT TCT ATG AAT TAT |
| NO: 8 | | CAG AAC GAC-3′ |
| SEQ ID | Ald-aroG4 RV2: | 5′-GTC GTT CTG ATA ATT CAT AGA AAT |
| NO: 9 | | AGG AGT CTT AGG-3′ |
| SEQ ID | Ald-trpE8 FW2: | 5′-CCT AAG ACT CCT ATT TCT ATG CAA |
| NO: 10 | | ACA CAA AAA CC-3′ |
| SEQ ID | Ald-trpE8 RV2: | 5′-GG TTT TTG TGT TTG CAT AGA AAT AGG |
| NO: 11 | | AGT CTT AGG-3 |
| SEQ ID | Ald-trpD FW2: | 5′-CCT CCT AAG ACT CCT ATT TCT ATG GCT |
| NO: 12 | | GAC ATT CTG CTG CTC-3′ |
| SEQ ID | Ald-trpD RV2: | 5′-GAG CAG CAG AAT GTC AGC CAT AGA |
| NO: 13 | | AAT AGG AGT CTT AGG AGG-3′ |
| SEQ ID | Linking Primer | 5′ACG ACG GCC AGT GCC AAG CTT CTG |
| NO: 14 | FW: | CAG GTC CCC AGA TTA GCC TTT TCA-3′ |
| SEQ ID | Linking Primer | 5′TGA AAA GGC TAA TCT GGG GAC CT TAT |
| NO: 15 | RV: | CTT TAA TCA TAT TCC-3′ |
| SEQ ID | aroG4_515-532F | 5′-ACC GCG AAC TGG CAT CAG-3′ |
| NO: 16 | | |
| SEQ ID | aroG4_572-555R | 5′-CCG TCG GTG CCA TTT TTG-3′ |
| NO: 17 | | |
| SEQ ID | trpE8_741-761F | 5′-CGT AGT GCG TTT GTT GCA AAA-3′ |
| NO: 18 | | |
| SEQ ID | trpE8_801-782R | 5′-AGA TGG CAC CAC CTG GAA AA-3′ |
| NO: 19 | | |
| SEQ ID | trpD_383-399F | 5′-CGG TGG CGC GTT ATC AC-3′ |
| NO: 20 | | |
| SEQ ID | trpD_446-427R | 5′-TGG GCG TTG ATG GTT AAA CC-3′ |
| NO: 21 | | |
| SEQ ID | ORCA4(introduced)_ | 5′-CAG TCT TTC TTC GGA ATC GTG TTC-3′ |
| NO: 22 | 189-212F | |
| SEQ ID | ORCA4(introduced)_ | 5′-CTG CAA GAC TCT GAG GAC AAT GAG-3′ |
| NO: 23 | 260-237R | |
| SEQ ID | ORCA4(endogenous)_ | 5′-TGA GGA ATG GAA GCG GTA CAG-3′ |
| NO: 24 | 351-371F | |
| SEQ ID | ORCA4(endogenous)_ | 5′-TCT CCG CCG CAA ATT TTC-3′ |
| NO: 25 | 412-395R | |
| SEQ ID | PAS FW | 5′-TGGGGTCTCTTCCTCTGTTG-3′ |
| NO: 33 | | |
| SEQ ID | PAS RV | 5′-AGAATTCTGCCTCTGGCATC-3′ |
| NO: 34 | | |
| SEQ ID | CS FW | 5′-TGGTTTCCATGATTTCAACG-3′ |
| NO: 35 | | |
| SEQ ID | CS RV | 5′-TCTCCGTTTCCAAGGTGAAG-3′ |
| NO: 36 | | |
| SEQ ID | TS FW | 5′-AGATGCTCCTGGTGGAAATG-3′ |
| NO: 37 | | |
| SEQ ID | TS RV | 5′-CTCTAGCTTCATCGGCAACC-3′ |
| NO: 38 | | |

The present disclosure makes it possible to produce alkaloids with higher efficiency than ever before. Therefore, the present disclosure is extremely useful industrially.

What is claimed is:

1. A method of producing an alkaloid, comprising:
expressing, using genetic engineering, a protein involved in tryptophan biosynthesis and a protein involved in secologanin biosynthesis in a plant of a genus Catharanthus in a family Apocynaceae.

2. A method of producing an alkaloid according to claim 1, wherein expressing comprises introducing a gene encoding the protein involved in tryptophan biosynthesis and a gene encoding the protein involved in secologanin biosynthesis into the plant of the genus Catharanthus in the family Apocynaceae.

3. The method according to claim 1, wherein expressing comprises introducing the gene encoding the protein involved in tryptophan biosynthesis and the gene encoding the protein involved in secologanin biosynthesis into the plant of the genus Catharanthus in the family Apocynaceae using a vector containing the genes.

4. The method according to claim 1, wherein expressing comprises introducing the gene encoding the protein involved in tryptophan biosynthesis and the gene encoding the protein involved in secologanin biosynthesis into a leaf and/or stem of Catharanthus roseus.

5. The method according to claim 1, wherein the protein involved in secologanin biosynthesis is ORCA4.

6. The method according to claim 1, wherein the protein involved in tryptophan biosynthesis contains at least one protein selected from the group consisting of AroG, TrpD, and TrpE.

7. The method according to claim 1, wherein the protein involved in tryptophan biosynthesis contains AroG, TrpD, and TrpE.

8. The method according to claim 7, wherein:
the AroG is an AroG protein modified to suppress feedback control of 3-deoxy-D-arabino-heptulosonate-7-phosphate synthase by phenylalanine; and/or
the TrpE is a TrpE protein modified to suppress feedback control of anthranilate synthase by tryptophan.

9. The method according to claim 7, wherein:
the AroG is an AroG protein obtained by expressing a gene having a DNA sequence of SEQ ID NO: 27; and/or
the TrpD is a TrpD protein obtained by expressing a gene having a DNA sequence of SEQ ID NO: 28; and/or
the TrpE is a TrpE protein obtained by expressing a gene having a DNA sequence of SEQ ID NO: 30.

10. The method according to claim 1, wherein the alkaloid is a vinca alkaloid.

11. The method according to claim 1, wherein the alkaloid is at least one alkaloid selected from the group consisting of tabersonine, vindoline, catharanthine, vinblastine, vincristine, ajmalicine, serpentine, stemmadenine, and strictosidine.

12. The method according to claim 1, wherein the alkaloid is tabersonine.

13. The method according to claim 1, wherein the alkaloid is stemmadenine.

14. The method according to claim 1, wherein the plant is Catharanthus roseus.

15. A vector comprising: a gene encoding a protein involved in tryptophan biosynthesis and a gene encoding a protein involved in secologanin biosynthesis.

16. An Agrobacterium comprising:
a vector containing a gene encoding a protein involved in tryptophan biosynthesis and a gene encoding a protein involved in secologanin biosynthesis; or
a mixture of vectors containing at least one gene selected from
the gene encoding the protein involved in tryptophan biosynthesis, and
the gene encoding the protein involved in secologanin biosynthesis;
wherein the mixture of vectors further contains
the gene encoding the protein involved in tryptophan biosynthesis, and
the gene encoding the protein involved in secologanin biosynthesis in the entire mixture.


SEQUENCE LISTING (41 sequences)

SEQ ID NO: 1 - DNA, 24 bp, Artificial Sequence
  primer
atgaattatcagaacgacgattta

SEQ ID NO: 2 - DNA, 24 bp, Artificial Sequence
  primer
ttacccgcgacgcgcttttactgc

SEQ ID NO: 3 - DNA, 24 bp, Artificial Sequence
  primer
atgcaaacacaaaaaccgactctc

SEQ ID NO: 4 - DNA, 24 bp, Artificial Sequence
  primer
tcagaaagtctcctgtgcatgatg

SEQ ID NO: 5 - DNA, 35 bp, Artificial Sequence
  primer
atggctgacattctgctgctcgataatatcgactc

SEQ ID NO: 6 - DNA, 33 bp, Artificial Sequence
  primer
ttaccctcgtgccgccagtgcggtgactctgtc

SEQ ID NO: 7 - DNA, 41 bp, Artificial Sequence
  primer
gggcatatggctgacattctgctgctcgataatatcgactc

SEQ ID NO: 8 - DNA, 36 bp, Artificial Sequence
  primer
cctaagactcctatttctatgaattatcagaacgac

SEQ ID NO: 9 - DNA, 36 bp, Artificial Sequence
  primer
gtcgttctgataattcatagaaataggagtcttagg

SEQ ID NO: 10 - DNA, 35 bp, Artificial Sequence
  primer
cctaagactcctatttctatgcaaacacaaaaacc

SEQ ID NO: 11 - DNA, 35 bp, Artificial Sequence
  primer
ggtttttgtgtttgcatagaaataggagtcttagg

SEQ ID NO: 12 - DNA, 42 bp, Artificial Sequence
  primer
cctcctaagactcctatttctatggctgacattctgctgctc

SEQ ID NO: 13 - DNA, 42 bp, Artificial Sequence
  primer
gagcagcagaatgtcagccatagaaataggagtcttaggagg

SEQ ID NO: 14 - DNA, 48 bp, Artificial Sequence
  primer
acgacggccagtgccaagcttctgcaggtccccagattagccttttca

SEQ ID NO: 15 - DNA, 41 bp, Artificial Sequence
  primer
tgaaaaggctaatctggggaccttatctttaatcatattcc

SEQ ID NO: 16 - DNA, 18 bp, Artificial Sequence
  primer
accgcgaactggcatcag

SEQ ID NO: 17 - DNA, 18 bp, Artificial Sequence
  primer
ccgtcggtgccatttttg

SEQ ID NO: 18 - DNA, 21 bp, Artificial Sequence
  primer
cgtagtgcgtttgttgcaaaa

SEQ ID NO: 19 - DNA, 20 bp, Artificial Sequence
  primer
agatggcaccacctggaaaa

SEQ ID NO: 20 - DNA, 17 bp, Artificial Sequence
  primer
cggtggcgcgttatcac

SEQ ID NO: 21 - DNA, 20 bp, Artificial Sequence
  primer
tgggcgttgatggttaaacc

SEQ ID NO: 22 - DNA, 24 bp, Artificial Sequence
  primer
cagtctttcttcggaatcgtgttc

SEQ ID NO: 23 - DNA, 24 bp, Artificial Sequence
  primer
ctgcaagactctgaggacaatgag

SEQ ID NO: 24 - DNA, 21 bp, Artificial Sequence
  primer
tgaggaatggaagcggtacag

SEQ ID NO: 25 - DNA, 18 bp, Artificial Sequence
  primer
tctccgccgcaaattttc

SEQ ID NO: 26 - DNA, 1053 bp, Escherichia coli
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 27 - DNA, 1053 bp, Escherichia coli
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccctacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 28 - DNA, 1596 bp, Escherichia coli
atggctgacattctgctgctcgataatatcgactcttttacgtacaacctggcagatcagttgcgcagcaatgggcataacgtggtgatttaccgcaaccatattccggcgcaaaccttaattgaacgcctggcgaccatgagcaatccggtgctgatgctttctcctggccccggtgtgccgagcgaagccggttgtatgccggaactcctcacccgcttgcgtggcaagctgcccattattggcatttgcctcggacatcaggcgattgtcgaagcttacgggggctatgtcggtcaggcgggcgaaattctccacggtaaagcctccagcattgaacatgacggtcaggcgatgtttgccggattaacaaacccgctgccggtggcgcgttatcactcgctggttggcagtaacattccggccggtttaaccatcaacgcccattttaatggcatggtgatggcagtacgtcacgatgcggatcgcgtttgtggattccagttccatccggaatccattctcaccacccagggcgctcgcctgctggaacaaacgctggcctgggcgcagcagaaactagagccagccaacacgctgcaaccgattctggaaaaactgtatcaggcgcagacgcttagccaacaagaaagccaccagctgttttcagcggtggtgcgtggcgagctgaagccggaacaactggcggcggcgctggtgagcatgaaaattcgcggtgagcacccgaacgagatcgccggggcagcaaccgcgctactggaaaacgcagcgccgttcccgcgcccggattatctgtttgctgatatcgtcggtactggcggtgacggcagcaacagtatcaatatttctaccgccagtgcgtttgtcgccgcggcctgtgggctgaaagtggcgaaacacggcaaccgtagcgtctccagtaaatctggttcgtccgatctgctggcggcgttcggtattaatcttgatatgaacgccgataaatcgcgccaggcgctggatgagttaggtgtatgtttcctctttgcgccgaagtatcacaccggattccgccacgcgatgccggttcgccagcaactgaaaacccgcaccctgttcaatgtgctggggccattgattaacccggcgcatccgccgctggcgttaattggtgtttatagtccggaactggtgctgccgattgccgaaaccttgcgcgtgctggggtatcaacgcgcggcggtggtgcacagcggcgggatggatgaagtttcattacacgcgccgacaatcgttgccgaactgcatgacggcgaaattaaaagctatcagctcaccgcagaagactttggcctgacaccctaccaccaggagcaactggcaggcggaacaccggaagaaaaccgtgacattttaacacgtttgttacaaggtaaaggcgacgccgcccatgaagcagccgtcgctgcgaacgtcgccatgttaatgcgcctgcatggccatgaagatctgcaagccaatgcgcaaaccgttcttgaggtactgcgcagtggttccgcttacgacagagtcaccgcactggcggcacgagggtaa

SEQ ID NO: 29 - DNA, 1563 bp, Escherichia coli
atgcaaacacaaaaaccgactctcgaactgctaacctgcgaaggcgcttatcgcgacaatcccaccgcgctttttcaccagttgtgtggggatcgtccggcaacgctgctgctggaatccgcagatatcgacagcaaagatgatttaaaaagcctgctgctggtagacagtgcgctgcgcattacagctttaggtgacactgtcacaatccaggcactttccggcaacggcgaagccctcctggcactactggataacgccctgcctgcgggtgtggaaagtgaacaatcaccaaactgccgtgtgctgcgcttcccccctgtcagtccactgctggatgaagacgcccgcttatgctccctttcggtttttgacgctttccgtttattgcagaatctgttgaatgtaccgaaggaagaacgagaagccatgttcttcggcggcctgttctcttatgaccttgtggcgggatttgaagatttaccgcaactgtcagcggaaaataactgccctgatttctgtttttatctcgctgaaacgctgatggtgattgaccatcagaaaaaaagcacccgtattcaggccagcctgtttgctccgaatgaagaagaaaaacaacgtctcactgctcgcctgaacgaactacgtcagcaactgaccgaagccgcgccgccgctgccagtggtttccgtgccgcatatgcgttgtgaatgtaatcagagcgatgaagagttcggtggcgtagtgcgtttgttgcaaaaagcgattcgcgctggagaaattttccaggtggtgccatctcgccgtttctctctgccctgcccgtcaccgctggcggcctattacgtgctgaaaaagagtaatcccagcccgtacatgttttttatgcaggataatgatttcaccctatttggcgcgtcgccggaaagctcgctcaagtatgatgccaccagccgccagattgagatctacccgattgccggaacacgcccacgcggtcgtcgcgccgatggttcactggacagagatctcgacagccgtattgaactggaaatgcgtaccgatcataaagagctgtctgaacatctgatgctggttgatctcgcccgtaatgatctggcacgcatttgcacccccggcagccgctacgtcgccgatctcaccaaagttgaccgttattcctatgtgatgcacctcgtctctcgcgtagtcggcgaactgcgtcacgatcttgacgccctgcacgcttatcgcgcctgtatgaatatggggacgttaagcggtgcgccgaaagtacgcgctatgcagttaattgccgaggcggaaggtcgtcgccgcggcagctacggcggcgcggtaggttatttcaccgcgcatggcgatctcgacacctgcattgtgatccgctcggcgctggtggaaaacggtatcgccaccgtgcaagcgggtgctggtgtagtccttgattctgttccgcagtcggaagccgacgaaacccgtaacaaagcccgcgctgtactgcgcgctattgccaccgcgcatcatgcacaggagactttctga

SEQ ID NO: 30 - DNA, 1563 bp, Escherichia coli
atgcaaacacaaaaaccgactctcgaactgctaacctgcgaaggcgcttatcgcgacaattccaccgcgctttttcaccagttgtgtggggatcgtccggcaacgctgctgctggaatccgcagatatcgacagcaaagatgatttagaaagcctgctgctggtagacagtgcgctgcgcattacagctttaggtgacactgtcacaatccaggcactttccggcaacggcgaagccctcctggcactactggataacgccctgcctgcgggtgtggaaagtgaacaatcaccaaactgccgtgtgctgcgcttcccccctgtcagtccactgctggatgaagacgcccgcttatgctccctttcggtttttgacgctttccgtttattgcagaatctgttgaatgtaccgaaggaagaacgagaagccatgttcttcggcggcctgttctcttatgaccttgtggcgggatttgaagatttaccgcaactgtcagcggaaaataactgccctgatttctgtttttatctcgctgaaacgctgatggtgattgaccatcagaaaaaaagcacccgtattcaggccagcctgtttgctccgaatgaagaagaaaaacaacgtctcactgctcgcctgaacgaactacgtcagcaactgaccgaagccgcgccgccgctgccagtggtttccgtgccgcatatgcgttgtgaatgtaatcagagcgatgaagagttcggtggcgtagtgcgtttgttgcaaaaagcgattcgcgctggagaaattttccaggtggtgccatctcgccgtttctctctgccctgcccgtcaccgctggcggcctattacgtgctgaaaaagagtaatcccagcccgtacatgttttttatgcaggataatgatttcaccctatttggcgcgtcgccggaaagctcgctcaagtatgatgccaccagccgccagattgagatctacccgattgccggaacacgcccacgcggtcgtcgcgccgatggttcactggacagagatctcgacagccgtattgaactggaaatgcgtaccgatcataaagagctgtctgaacatctgatgctggttgatctcgcccgtaatgatctggcacgcatttgcacccccggcagccgctacgtcgccgatctcaccaaagttgaccgttattcctatgtgatgcacctcgtctctcgcgtagtcggcgaactgcgtcacgatcttgacgccctgcacgcttatcgcgcctgtatgaatatggggacgttaagcggtgcgccgaaagtacgcgctatgcagttaattgccgaggcggaaggtcgtcgccgcggcagctacggcggcgcggtaggttatttcaccgcgcatggcgatctcgacacctgcattgtgatccgctcggcgctggtggaaaacggtatcgccaccgtgcaagcgggtgctggtgtagtccttgattctgttccgcagtcggaagccgacgaaacccgtaacaaagcccgcgctgtactgcgcgctattgccaccgcgcatcatgcacaggagactttctga

SEQ ID NO: 31 - DNA, 174 bp, Catharanthus roseus
atggctatgtctaatacttctgctcttgcttctaagcttcttccttcttgtaagcctcatcaacctactcttacttttttttctccttctactacttgtcaaaagaagcctagatcttctagacctatttctgctgctgttcatgttactcaacctcctaagactcctatttct

SEQ ID NO: 32 - DNA, 675 bp, Catharanthus roseus
atgcttgacgaaattactatatcagcaagtgatcttgcattactttccttcattgaagaacatctactctctgattcatcagaaatattgccaaattcggatgatgacgattcatccaatcatctaatagatagctcatgggaggaaatcctgctgaagtcgattctggaaccagaacctcctgctggcagtctttcttcggaatcgtgttcaaacagtaccgctacagcggccggctcattgtcctcagagtcttgcagtaattcgactgccacagctgctggaagtccttctagcgagtcttgttgcaattctagtacagcggctgaatcatcgagtgaagaaaacagcgaagaatggaaaaggtatagaggagtgcggaggcgcttgtggggcaagtttgccgcagaaattagagatccaaataaaaaaggctcgcgaatttggcttgggacctatgaaactcctgaagatgcggctcttgcatacgatggtgctgcttttaagatgaggggctcaaaagcaaaactcaattttccacacttgattgggtcaaatattagtgagccggttcgagtaaacccaaggaaacgctataggaaattaactacaacttctagcagctattccagtgaagaggacaatgaaggtcgtaaaaagcgtcgaaattaatga

SEQ ID NO: 33 - DNA, 20 bp, Artificial Sequence
  primer
tggggtctcttcctctgttg

SEQ ID NO: 34 - DNA, 20 bp, Artificial Sequence
  primer
agaattctgcctctggcatc

SEQ ID NO: 35 - DNA, 20 bp, Artificial Sequence
  primer
tggtttccatgatttcaacg

SEQ ID NO: 36 - DNA, 20 bp, Artificial Sequence
  primer
tctccgtttccaaggtgaag

SEQ ID NO: 37 - DNA, 20 bp, Artificial Sequence
  primer
agatgctcctggtggaaatg

SEQ ID NO: 38 - DNA, 20 bp, Artificial Sequence
  primer
ctctagcttcatcggcaacc

SEQ ID NO: 39 - DNA, 294 bp, Catharanthus roseus
tggaattgtgaagacgatgaatcatcggaagaatatatcgatggattgggaaggcttgaggaattaatgactccatatgtgaaacaaccaagaggttcttggttcagcaccagaaacctttataccggtaaaaataaaggtccaggaacaacttattccaaagctaaagaatggggatttcggtattttaataataatttcaaaaagttggcccttatcaaaggacaagttgatccagaaaacttcttctactatgaacaaagcattccccctctccatttacaagtcgaactt

SEQ ID NO: 40 - DNA, 254 bp, Catharanthus roseus
tacccgcagcttatgaagattgttgggaagcccttcaatgggttgcttctcatgtgggtctcgacaattccggcctaaagacagctattgataaagatccatggataataaactatggtgatttcgatagactgtatttggcgggtgacagtcctggtgctaatattgttcacaacacacttatcagagctggaaaagagaaactgaagggcggagtgaaaattttgggggcaattctttactacccatatttc

SEQ ID NO: 41 - DNA, 257 bp, Catharanthus roseus
tacccgcagcttatgaagattgctgggaagcccttcaatgggttgcttctcatgtgggtctcgacatttccagcttgaagacatgtattgataaagatccatggataatcaactatgccgatttcgatagactctatttgtggggtgatagcaccggtgccaatattgttcacaacacacttatcagatctggtaaagagaaattgaacggcggcaaagtgaagattttgggggcaattctttactacccatatttc